(19) United States
(12) Patent Application Publication
(10) Pub. No.: US 2022/0159945 A1
(43) Pub. Date: May 26, 2022

(54) CRYOPRESERVATION AND CELL CULTURE MEDIA

(71) Applicant: Akron Biotechnology, LLC, Boca Raton, FL (US)

(72) Inventor: Claudia Zylberberg, Delray Beach, FL (US)

(21) Appl. No.: 17/600,928

(22) PCT Filed: Apr. 3, 2020
(86) PCT No.: PCT/US2020/026598

§ 371 Date: Oct. 1, 2021

Related U.S. Application Data
(60) Provisional application No. 62/828,867, filed on Apr. 3, 2019.

(51) Int. Cl. A01N 1/02; C12N 5/00

(52) U.S. Cl. CPC A01N 1/0226; C12N 2501/315; C12N 2501/2317; C12N 2501/2321; C12N 2501/2322; C12N 2501/22; C12N 2501/2302; C12N 2501/2312; C12N 2501/2318; C12N 2501/2327; C12N 2501/24; C12N 2501/235; C12N 2501/13; C12N 2501/145; C12N 2501/25; C12N 2501/155; C12N 5/0018

(57) ABSTRACT

Cell culture media, preservative media or cryopreservation media include a low dose of one or more cytokines, e.g. interleukin-2 (IL-2).

### CROSS REFERENCE TO RELATED APPLICATIONS

This application claims the benefit of U.S. Provisional Application 62/828,867 filed on Apr. 3, 2019. The entire contents of this application are incorporated herein by reference in their entirety.

### FIELD OF THE INVENTION

The present invention is directed to cryopreservation and cell culture media which include low-dose cytokines and other related factors.

### BACKGROUND

Cytokines are a class of signaling molecules or intercellular mediators (proteins, peptides, and glycoproteins) that primarily affect cells of the immune system; however, they can affect various cell types outside of the immune system. They differ from classical hormones in that they are produced by a number of tissue or cell types rather than by specialized glands. The effects of cytokines on cells can yield different outcomes. Some cytokines cause cell proliferation, while others may cause chemotaxis between cell types, and others can even cause cell death. Cytokines and growth factors are similar in their structure and mechanism of action. Both bind and initiate signaling pathways, and many share several intracellular signaling components.

### SUMMARY

Embodiments of the invention are directed to cell culture media, preservative media or cryopreservative media comprising a low dose of one or more: cytokines, hormones, mitogens or combinations thereof. Addition of one or more cytokines to the cell culture media, preservative media or cryopreservative media provide the stimulus needed to enhance the cell proliferation in culture, especially during thawing of the cells. This is important as the lag time in the culturing and proliferation of cells from thawing to obtaining numbers of cells needed for, e.g., immunotherapy, is decreased.

In certain embodiments, the cell culture medium, the preservative medium or the cryopreservative medium comprises one or more cytokines, modified cytokines, mutants or derivatives thereof. In this and other embodiments, the cytokines comprise one or more type 1 cytokines, type 2 cytokines, type 17 cytokines or combinations thereof. In these and other embodiments, the type 1 cytokines comprise: interleukin IL-2, IL-3, IL-4, IL-5, IL-6, IL-7, IL-9, IL-11, IL-18, IL-27, interferon-gamma (IFNγ), granulocyte macrophage-colony stimulating factor (GM-CSF), granulocyte colony stimulating factor (G-CSF), leukemia inhibiting factor (LIF), ciliary neurotrophic factor (CNTF), thrombopoietin, tumor necrosis factor alpha (TNFα) growth hormone, prolactin or combinations thereof. In these and other embodiments, the type 2 cytokines comprise: transforming growth factor beta (TGF-β), interleukin IL-4, I L-5, IL-9, IL-10, IL-13, IL-25, transforming growth factor beta (TGF-β) or combinations thereof. In these and other embodiments, the type 17 cytokines comprise: interleukin (IL)-17, IL-21, IL-22 or combinations thereof. In these and other embodiments, the cytokine is a hematopoietic cytokine comprising: granulocyte macrophage colony stimulating factor (GM-CSF), granulocyte colony stimulating factor (G-CSF), macrophage colony stimulating factor (M-CSF), erythropoietin (EPO), thrombopoietin (TPO), stem cell factor (SCF), stem cell growth factor (SCGF), bone morphogenetic protein (BMP) or combinations thereof.

In certain embodiments, preservation or cryopreservation medium comprising a low dose of one or more: cytokines, hormones, mitogens or combinations thereof. In certain embodiments, the one or more cytokines comprise: interleukins, chemokines, growth factors, interferons, or combinations thereof. In certain embodiments, the one or more interleukins comprise: interleukins 1-40 (IL-1 to IL-40) or combinations thereof. In certain embodiments, the medium comprises an amount of one or more cytokines from about 1×10² International Units/ml to about 1×10⁹ International Units/ml.

In certain embodiments, the cytokine is IL-2, modified IL-2, IL-2 derivatives, IL-2 mutants or combinations thereof.

In certain embodiments, the cytokine is IL-15, modified IL-15, IL-15 derivatives, IL-15 mutants or combinations thereof.

In these and other embodiments, a low dose comprises from about 0.01 ng to about 100 mg per 100 ml. In these and other embodiments, a low dose comprises from about 0.01 ng to about 10 mg per 100 ml. In these and other embodiments, a low dose comprises from about 0.01 ng to about 1 mg per 100 ml. In these and other embodiments, a low dose comprises from about 0.01 ng to about 1 mg per 100 ml.

In certain embodiments, a low dose of a cytokine comprises between 0.001% and 10.0% v/v of the cell culture medium, preservative medium or cryopreservative medium. In certain embodiments, a low dose of a cytokine comprises between 0.2% and 10.0% v/v or between 0.2% and 5.0% v/v of the cell culture medium, preservative medium or cryopreservative medium.

In certain embodiments, the cell culture medium, preservative medium or cryopreservative medium comprises from 0.001 μg/ml to 100.0 μg/ml of one or more cytokine(s), or from 0.001 μg/ml to about 75 μg/ml of a cytokine(s) or from 0.001 μg/ml to about 50.0 μg/ml, or from 0.001 μg/ml to about 25.0 μg/ml of a cytokine(s), or from 0.001 μg to about 15.0 μg/ml of a cytokine(s), or from 0.001 μg to about 10.0 μg/ml of a cytokine(s). In certain embodiments, the cell culture medium, preservative medium or cryopreservative medium comprises a concentration of one or more cytokines ranging from 0.001-100.00 μg/ml; 0.001-75.0 μg/ml; 0.001-50.0 μg/ml; 0.001-25.0 μg/ml; 0.001-15.0 μg/ml; 0.001-10.0 μg/ml; 0.001-5.0 μg/ml; 0.001-2.5 μg/ml; 0.001-1.0 μg/ml; 0.001-0.5 μg/ml; 0.001-0.30 μg/ml; 0.001-0.25 μg/ml; 0.001-0.10 μg/ml; 0.001-0.05 μg/ml; 0.01-100.0 μg/ml; 0.01-75.0 μg/ml; 0.01-50.0 μg/ml; 0.01-25.0 μg/ml; 0.01-15.0 μg/ml; 0.01-10.0 μg/ml; 0.01-5.0 μg/ml; 0.01-2.5 μg/ml; 0.01-1.0 μg/ml; 0.01-0.5 μg/ml; 0.01-0.30 μg/ml; 0.01-0.25 μg/ml; 0.01-0.10 μg/ml; 0.01-0.05 μg/ml; 0.05-100.0 μg/ml; 0.05-50.0 μg/ml; 0.05-75.0 μg/ml; 0.05-50.0 μg/ml; 0.05-25.0 μg/ml; 0.05-15.0 μg/ml; 0.05-10.0 μg/ml; 0.05-5.0 μg/ml; 0.05-2.5 μg/ml; 0.05-1.0 μg/ml; 0.05-0.5 μg/ml; 0.05-0.30 μg/ml; 0.05-0.25 μg/ml; 0.05-0.10 μg/ml; 0.05-0.05 μg/ml; 0.15-100.0 μg/ml; 0.15-75.0 μg/ml; 0.15-50.0 μg/ml; 0.15-25.0 μg/ml; 0.15-15.0 μg/ml; 0.15-10.0 μg/ml; 0.15-5.0 μg/ml; 0.15-2.5 μg/ml; 0.15-1.0 μg/ml; 0.15-0.5 μg/ml; 0.15-0.30 μg/ml; 0.15-0.25 μg/ml; 0.15-0.10 μg/ml; and 0.15-0.05 μg/ml.

In certain embodiments, the cell culture medium, preservative medium or cryopreservative medium comprises a cytokine(s) in a concentration ranging from 0.001-100 ng/ml; 0.001-90 ng/ml; 0.001-75 ng/ml; 0.001-50 ng/ml; 0.001-25 ng/ml; 0.01-100 ng/ml; 0.01-90 ng/ml; 0.01-75 ng/ml; 0.01-50 ng/ml; 0.01-25 ng/ml; 0.1-100 ng/ml; 0.1-90 ng/ml; 0.1-75 ng/ml; 0.1-50 ng/ml; 0.1-25 ng/ml; 1-75 ng/ml; 1-50 ng/ml; 1-25 ng/ml; 1-15 ng/ml; 1-10 ng/ml; 10-100 ng/ml; 10-75 ng/ml; 10-50 ng/ml; 10-25 ng/ml; 10-15 ng/ml; 15-100 ng/ml; 15-75 ng/ml; 15-50 ng/ml; 15-25 ng/ml; 15-20 ng/ml; 20-100 ng/ml; 20-75 ng/ml; 20-25 ng/ml; 25-100 ng/ml; 25-75 ng/ml; or 25-50 ng/ml.

In certain embodiments, the cell culture medium, preservative medium or cryopreservative medium comprises a cytokine(s) in a concentration ranging from 1×10² to about 1×10⁹ International Units/ml; 1×10³ units to 5×10⁸ International Units/ml; 1×10³ units to 1×10⁷ International Units/ml; 1×10³ units to 5×10⁷ International Units/ml; 1×10³ units to 1×10⁷ International Units/ml; 1×10³ units to 5×10⁶ International Units/ml; 1×10³ units to 1×10⁶ International Units/ml; 1×10³ units to 5×10⁵ International Units/ml; 1×10³ units to 1×10⁵ International Units/ml; 1×10³ units to 5×10⁴ International Units/ml; 1×10³ units to 1×10⁴ International Units/ml; or 1×10³ units to 5×10³ International Units/ml.

In these and other embodiments, any type of cell, including prokaryotic cells, tissues or biological sample may be preserved or cultured in the cell culture medium, preservative medium or cryopreservative medium.

Other aspects are described infra.

### Definitions

Unless otherwise defined, all terms (including technical and scientific terms) used herein have the same meaning as commonly understood by one of ordinary skill in the art to which this disclosure pertains. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The terminology used herein is for the purpose of describing particular embodiments only and is not intended to be limiting of the disclosure. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude, preferably within 5-fold, and also preferably within 2-fold, of a value. Where particular values are described in the application and claims, unless otherwise stated the term “about” meaning within an acceptable error range for the particular value should be assumed.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

A “biological medium” as used herein, is any type of medium that is used to grow, culture and maintain organs, tissues, cells etc., in vitro. A biological medium also encompasses any biocompatible agent, any pharmaceutical excipient, pharmaceutically and physiologically acceptable fluids such as water, physiological saline, balanced salt solutions, aqueous dextrose, glycerol or the like as a vehicle, tissue or organ culture media, any agent that can be administered in vivo to a subject, any agent that can be used in assays or for diluting or maintaining a biological sample, e.g. nucleic acids, peptides etc.

As used herein, a “biological sample” refers to a sample including tissues, cells, organs, biological fluids, polypeptides, nucleic acids, or other biological substances. In some embodiments a biological sample can further include preservatives. In some embodiments, a sample can be obtained from a subject. In some embodiments a sample can be a diagnostic sample obtained from a subject. By way of non-limiting example, a sample can be a prokaryotic cell, a gamete, sperm, eggs, an embryo, a zygote, chondrocytes, red blood cells, blood, portions or fractions of blood, hepatic cells, fibroblasts, stem cells, cord blood cells, adult stem cells, Mesenchymal Stem cells or synonymously ‘marrow stromal cells’, ‘bone marrow (BM) stromal cells’ and ‘mesenchymal stromal cells’, induced pluripotent stem cells, autologous cells, autologous stem cells, bone marrow cells, hematopoietic cells, hematopoietic stem cells, somatic cells, germ line cells, differentiated cells, somatic stem cells, embryonic stem cells, serum, plasma, sputum, cerebrospinal fluid, urine, tears, alveolar isolates, pleural fluid, pericardial fluid, cyst fluid, tumor tissue, normal tissues, organs, a biopsy, saliva, an aspirate, or combinations thereof. In some embodiments, a sample can be obtained by resection, biopsy, or egg retrieval.

The term “chimeric antigen receptor” or “CAR” as used herein refers to an antigen-binding domain that is fused to an intracellular signaling domain capable of activating or stimulating an immune cell, and in certain embodiments, the CAR also comprises a transmembrane domain. In certain embodiments the CAR's extracellular antigen-binding domain is composed of a single chain variable fragment (scFv) derived from fusing the variable heavy and light regions of a murine or humanized monoclonal antibody. Alternatively, scFvs may be used that are derived from Fab's (instead of from an antibody, e.g., obtained from Fab libraries). In various embodiments, the scFv is fused to the transmembrane domain and then to the intracellular signaling domain. “First-generation” CARs include those that solely provide CD3 signals upon antigen binding, “Second-generation” CARs include those that provide both co-stimulation (e.g., CD28 or CD137) and activation (CD3). “Third-generation” CARs include those that provide multiple co-stimulation (e.g. CD28 and CD137) and activation (CD3). A fourth generation of CARs have been described, CAR T cells redirected for universal cytokine killing (TRUCKS) where the vector containing the CAR construct possesses a cytokine cassette. When the CAR is ligated, the CAR T cell deposits a pro-inflammatory cytokine into the tumor lesion. A CAR-T cell is a T cell that expresses a chimeric antigen receptor. The phrase “chimeric antigen receptor (CAR),” as used herein and generally used in the art, refers to a recombinant fusion protein that has an antigen-specific extracellular domain coupled to an intracellular domain that directs the cell to perform a specialized function upon binding of an antigen to the extracellular domain. The terms “artificial T-cell receptor,” “chimeric T-cell receptor,” and “chimeric immunoreceptor” may each be used interchangeably herein with the term “chimeric antigen receptor.”

As used herein, the terms “cell,” “cell line,” and “cell culture” may be used interchangeably. All of these terms also include their progeny, which are any and all subsequent generations. It is understood that all progeny may not be identical due to deliberate or inadvertent mutations. The “cells” can be prokaryotic or eukaryotic and encompass all species, e.g. mammals, fish, birds, reptiles, insects, fungi, yeast, bacterial and the like. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

By “cell culture” or “culture” is meant the maintenance of cells in an artificial, in vitro environment. It is to be understood, however, that the term “cell culture” is a generic term and may be used to encompass the cultivation not only of individual cells, but also of tissues, organs, organ systems or whole organisms, for which the terms “tissue culture,” “organ culture,” “organ system culture” or “organotypic culture” may occasionally be used interchangeably with the term “cell culture.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“Cryopreserved cells” or “cryopreserved tissues” are cells or tissues that have been preserved by cooling to a sub-zero temperature. Cryopreserved cells include eukaryotic and prokaryotic cells, including pluripotent stem cells, immune cells, blood cells, transformed cells, transfected cells, CAR-T cells, TIL cells, NK cells or any type of cell useful for transfusing or transplanting into a subject, or for diagnostic and research purposes. Cryopreserved cells and tissues include, for example, animal, insect, bird, fish, reptile and plant cells or tissues.

As used herein, the phrase “cryopreservative composition” refers to a chemical or a chemical solution which facilitates the process of cryopreservation by reducing the injury of cells and tissues during freezing and thawing. The cryopreservative compositions protect cells and tissues from damage associated with storage at sub-zero temperature and/or freezing, e.g., cell membrane damage due to ice crystal formation. The term “preservative” media or composition refers to a chemical or a chemical solution which allows the long-term storage and/or culturing of cells at varying temperatures, including room temperature, refrigeration, typical cell-culturing temperatures, freezing temperatures and the like. The compositions of the present disclosure are thus cryoprotective, cryopreservative, preservative, or combinations thereof.

As used herein, the term “immune cells” generally includes white blood cells (leukocytes) which are derived from hematopoietic stem cells (HSC) produced in the bone marrow “Immune cells” includes, e.g., lymphocytes (T cells, B cells, natural killer (NK) cells) and myeloid-derived cells (neutrophil, eosinophil, basophil, monocyte, macrophage, dendritic cells). Among the sub-types and subpopulations of T cells and/or of CD4⁺ and/or of CD8⁺ T cells are naive T (T_{N}) cells, effector T cells (T_{EFF}), memory T cells and sub-types thereof, such as stem cell memory T (T_{SCM}), central memory T (T_{CM}) effector memory T (T_{EM}), or terminally differentiated effector memory T cells (T_{EMRA}), tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as T_{H}1 cells, T_{H}2 cells, T_{H}3 cells, T_{H}17 cells, T_{H}9 cells, T_{H}22 cells, follicular helper T (Tfh) cells, alpha/beta T cells (αβ T), and gamma/delta T (γδ T) cells.

The term “immune effector cell,” as used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NK-T) cells, mast cells, and myeloic-derived phagocytes. “Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “organ” as used herein refers to a structure of bodily tissue in a subject, e.g., a mammalian subject such as a human, wherein the tissue structure as a whole is specialized to perform a particular bodily function. Organs which are transplanted within the meaning of the present invention include for example, but without limitation, cornea, skin, heart, lung, kidney, pancreas, liver, spleen. In some embodiments, the term “organ” also encompasses decellularized and recellularized organs, as well as engineered and artificial organs and tissues, including engineered organs (e.g., tissue engineered constructs), engineered organs comprising a bioscaffold, tissues, organ slices and partial organs.

As used in this specification and the appended claims, the term “or” is generally employed in its sense including “and/or” unless the content clearly dictates otherwise.

“Pharmaceutical agent,” also referred to as a “drug,” or “therapeutic agent” is used herein to refer to an agent that is administered to a subject to treat a disease, disorder, or other clinically recognized condition that is harmful to the subject, or for prophylactic purposes, and has a clinically significant effect on the body to treat or prevent the disease, disorder, or condition. Therapeutic agents include, without limitation, agents listed in the United States Pharmacopeia (USP), Goodman and Gilman's The Pharmacological Basis of Therapeutics, 12^{th} Ed., McGraw Hill, 2001; Katzung, B. (ed.) Basic and Clinical Pharmacology, McGraw-Hill/Appleton and Lange; 8^{th} edition (Sep. 21, 2000); Physician's Desk Reference (Thomson Publishing), and/or The Merck Manual of Diagnosis and Therapy, 18^{th} ed. (2006), or the 19^{th} ed (2011), Robert S. Porter, MD., Editor-in-chief and Justin L. Kaplan, MD., Senior Assistant Editor (eds.), Merck Publishing Group, or, in the case of animals, The Merck Veterinary Manual, 10^{th} ed., Cynthia M. Kahn, B. A., M. A. (ed.), Merck Publishing Group, 2010.

As used herein, the term “preservative compositions” include compositions embodied herein, wherein the biological sample is stored over short e.g. 1 day and over extended periods of time e.g. weeks, months or years at temperatures from about −150° C. to room temperature (e.g. around 20-22° C.) or ambient temperatures or up to 30° C. or up to 37° C. or up to 40° C. and the like. In certain embodiments, the temperatures range from −150° C. to about 0° C. In certain embodiments, the temperatures range from −70° C. to about 0° C. In certain embodiments, the temperatures range from −70° C. to about 20° C. In certain embodiments, the temperatures range from 0° C. to about 30° C.

As used herein, the term “stem cell” refers to those cells which retain the ability to renew themselves through mitotic cell division and which can differentiate into a diverse range of specialized cell types. It includes both embryonic stem cells that are found in blastocysts, and adult stem cells that are found in adult tissues. “Totipotent cells” refers to cells that have the ability to develop into cells derived from all three embryonic germ layers (mesoderm, endoderm and ectoderm) and an entire organism (e.g., human being if placed in a woman's uterus in the case of humans). Totipotent cells may give rise to an embryo, the extra embryonic membranes and all post-embryonic tissues and organs. The term “pluripotent” as used herein is intended to mean the ability of a cell to give rise to differentiated cells of all three embryonic germ layers. “Multipotent cells” refers to cells that can produce only cells of a closely related family of cells (e.g. hematopoietic stem cells differentiate into red blood cells, white blood cells, platelets, etc.). “Unipotent cells” refers to cells that have the capacity to develop/differentiate into only one type of tissue/cell type (e.g. skin cells).

The term “viability” as used herein refers to the state of a cell or a tissue. The cells or tissues may have undergone multiple freeze-thaw cycles in the compositions embodied herein. Viability of cells is also easily determined, for example, immunostaining, dye exclusion, metabolic tests etc. The term “viability” also refers to the state of, a tissue or an organ's survival capability, e.g., capable of survival after transplantation into a recipient. Viability can be used as a measure of the entire organ's survival or a part of the organ, or the viability of cells within the organ. The term “viability” also includes reference to cells, cell cultures, tissues, etc.

It is to be understood that compounds embodied herein that have varying molecular weights are included. For example, dextran −4, −150 etc. Polysucrose 20, −1000 etc.

### DETAILED DESCRIPTION

### Cell Culture, Preservative and Cryopreservative Media

Cryopreservation involves the storage of biological samples, including cells, tissues, and organs, at sub-zero temperatures at which biological activity effectively ceases. This allows storage of biological samples with minimal degradation of the sample and/or long-term storage of biological samples. Cryopreservation can be performed in a variety of different manners. For example, cryopreservation can be performed at a slower rate, referred to herein as “slow-rate cryopreservation,” wherein the decrease in temperature of the biological sample to sub-zero temperatures is typically performed over minutes, hours, days, etc. As another example, cryopreservation can be performed at a faster rate of cryopreservation, referred to herein as “fast-rate cryopreservation” which includes for example, vitrification and/or ultra-rapid freezing, wherein the decrease in temperature of the biological sample to sub-zero temperatures is typically performed in seconds or fractions of a second, such as milliseconds and at temperatures significantly lower than the temperatures associated with slow-rate cryopreservation. In embodiments, the slow-rate cryopreservation process may occur at temperatures ranging from 0° C. to −150° C. whereas fast-rate cryopreservation processes may occur at temperatures lower temperatures than −150° C.

The cryopreservative compositions described herein may be adopted for use in any type of cryopreservation method, including for example slow-rate cryopreservation, or fast-rate cryopreservation including vitrification, and/or ultra-rapid freezing. These compositions can also be used in long-term storage and/or culturing of cells without the need for freezing. Accordingly, long term storage, lasting years and without freezing, can include maintaining the cells at temperatures typical for storing or culturing. For example, from −150° C. up to 37° C., 40° C. etc., depending on the cell-type.

The cryopreservative medium can include any type of molecule or cell that is desired to be preserved over long periods of time and still retain activity or function. In certain embodiments, the cryopreservative media comprise cytokines, growth factors, enzymes, hormones, therapeutic agents, peptides, antibodies, oligonucleotides, small molecules or combinations thereof.

In certain embodiments, the cell culture medium, the preservative medium or the cryopreservative medium comprises one or more cytokines, modified cytokines, mutants or derivatives thereof. Cytokines are a cell-signaling group of low molecular weight extracellular polypeptides/glycoproteins synthesized by different immune cells, mainly, by T cells, neutrophils and macrophages, which are responsible to promote and regulate immune response (i.e. activity, differentiation, proliferation and production of cells and other cytokines). These polypeptides act on signaling molecules and cells, stimulating them toward sites of inflammation, infections, traumas, acting on primary lymphocyte growth factors and other biological functions. Cytokines may act in the site where they are produced (autocrine action), in nearby cells (paracrine action) or in distant cells (endocrine action). In this sense, they are important in the development and regulation of immune system cells. There are many types of cytokines, including chemokines, interferons, interleukins, lymphokines, tumor necrosis factor but generally not hormones or growth factors (despite some terminologic overlap). All those types of cytokines are produced by a broad range of cells, including immune cells like macrophages, B lymphocytes, T lymphocytes and mast cells, as well as endothelial cells, fibroblasts, and various stromal cells; a given cytokine may be produced by more than one type of cell.

In this and other embodiments, the cytokines comprise one or more type 1 cytokines, type 2 cytokines, type 17 cytokines or combinations thereof. Type-1 cytokines are cytokines produced by Th1 T-helper cells while Type-2 cytokines are those produced by Th2 T-helper cells. In these and other embodiments, the type 1 cytokines comprise: include IL-2 (IL2), IFN-gamma (IFNγ), IL-12 (IL12) and TNF-β (TNFβ), while Type 2 cytokines include IL-4 (IL4), IL-5 (IL5), IL-6 (IL6), IL-10 (IL10), and IL-13 (IL13).

In these and other embodiments, the type 17 cytokines comprise: interleukin IL-17, IL-21, IL-22 or combinations thereof. In these and other embodiments, the cytokine is a hematopoietic cytokine comprising: granulocyte macrophage colony stimulating factor (GM-CSF), granulocyte colony stimulating factor (G-CSF), macrophage colony stimulating factor (M-CSF), erythropoietin (EPO), thrombopoietin (TPO), stem cell factor (SCF), stem cell growth factor (SCGF), bone morphogenetic protein (BMP) or combinations thereof.

These cytokines, growth factors, enzymes etc., can be included in the compositions here in varying doses. In certain embodiments, the cytokines, growth factors and the like are present in the cryopreservative medium in a low dose. a low dose comprises from about 0.01 ng to about 100 mg per 100 ml. In these and other embodiments, a low dose comprises from about 0.01 ng to about 10 mg per 100 ml. In these and other embodiments, a low dose comprises from about 0.01 ng to about 1 mg per 100 ml. In these and other embodiments, a low dose comprises from about 0.01 ng to about 1 mg per 100 ml.

In certain embodiments, a low dose of a cytokine comprises between 0.001% and 10.0% v/v of the cell culture medium, preservative medium or cryopreservative medium. In certain embodiments, a low dose of a cytokine comprises between 0.2% and 10.0% v/v or between 0.2% and 5.0% v/v of the cell culture medium, preservative medium or cryopreservative medium.

In certain embodiments, the cell culture medium, preservative medium or cryopreservative medium comprises a cytokine(s) in a concentration ranging from 1×10² to about 1×10⁹ International Units/ml; 1×10³ units to 5×10⁸ International Units/ml; 1×10³ units to 1×10⁷ International Units/ml; 1×10³ units to 5×10⁷ International Units/ml; 1×10³ units to 1×10⁷ International Units/ml; 1×10³ units to 5×10⁶ International Units/ml; 1×10³ units to 1×10⁶ International Units/ml; 1×10³ units to 5×10⁵ International Units/ml; 1×10³ units to 1×10⁵ International Units/ml; 1×10³ units to 5×10⁴ International Units/ml; 1×10³ units to 1×10⁴ International Units/ml; or 1×10³ units to 5×10³ International Units/ml.

In certain embodiments, the cell culture medium, preservative medium or cryopreservative medium comprises at last two or more cytokine(s) or other agents. For example, therapeutic agents, growth factors, hormones, and the like.

In certain embodiments, one or more or a combination of agents can be added to the preserved or stored biological sample, e.g. a lymphocyte, post-thawing. In certain embodiments, these agents can also be added to the culture medium when the biological sample is thawed and cultured to proliferate and expand. For example, cytokines such as IL7+ IL15 (50 ng/ml) which may be added to the cell culture media to boost the cell growth after post-thaw. In certain embodiments, these cytokines can be added in low doses to the preservative medium or cryopreservative medium based the culturing conditions needed for post-thaw growth of that cell or biological sample. For example, if the biological sample is isolated or cryopreserved peripheral blood mononuclear cells (PBMC) these IL-7 and IL-15 is added to the post-thaw culture conditions to increase the number of cells (W. Jennes et al. Journal of Immunological Methods 270 (2002) 99-108, incorporated by reference in its entirety). In certain embodiments, antigens or antigen presenting cells plus antigen can be added to the culture medium to select antigen specific lymphocytes, e.g. CD4⁺ T cells, CD8⁺ T cells. In certain embodiments, the cryopreservative medium for storing these cells can contain, for example IL-7 and IL-15 in varying doses.

Interferons

The interferon (IFN) family represents a widely expressed group of cytokines. It includes three main classes, designated as type I IFNs, type II IFN and type III IFNs. The two main type I IFNs includes IFN-α (further classified into 13 different subtypes such as IFN-α1, -α2, -α4, -α5, -α6, -α7, -α8, -α10, -α13, -α14, -α16, -α17 and -α21), and IFN-β. Type I IFNs present a potent antiviral effect by inhibiting viral replication, increasing the lysis potential of natural killer (NK) cells and the expression of MHC class I molecules on virus-infected cells, and stimulating the development of Th1 cells. During an infectious process, this type of interferon becomes abundant and is easily detectable in the blood. On the other hand, type II IFN has only one representative, IFN-γ. This cytokine plays a major role is macrophage activation both in innate and adaptive immune responses. Type III IFNs, also denoted IL-28/29, present similar biological effects to type I IFN, playing an important role in host defense against viral infections (Vinicius L. Ferreira, Helena H. L. Borba, Aline de F. Bonetti, Leticia P. Leonart and Roberto Pontarolo (Mar. 13, 2018). Cytokines and Interferons: Types and Functions, Autoantibodies and Cytokines, Wahid Ali Khan, IntechOpen, DOI: 10.5772/intechopen.74550).

Tumor Necrosis Factor (TNF)

The TNF superfamily and the TNF receptor superfamily, which has more than 40 members, being the most outstanding TNF-α (commonly named as TNF) and TNF-β (also named lymphotoxin), but also including cytokines and membrane proteins that have similar sequence homologies and a homotrimeric pyramidal structure (e.g., CD40 ligand, FAS ligand, OX40 ligand, GITR ligand and other several proteins). The binding of this family of cytokines with their respective receptors triggers especially inflammatory reactions.

TNFα is produced by different cells from the immune system, which includes T cells, NK cells, macrophages and monocytes. The stimulus for TNF expression includes different factors, such as bind to pathogen lipopolysaccharide (LPS) and other parts with toll-like receptors (TLRs), and also by other cytokines, highlighting IFNγ.

TNFβ, a type II transmembrane protein, is an important key in the development of lymph nodes and Peyer's patches, and also for the maintenance of secondary lymphoid organs. The expression of TNFβ is mainly stimulated by lymphocytes.

Interleukins

Interleukins (ILs) are a group of secreted proteins with diverse structures and functions. These proteins bind to receptors and are involved in the communication between leukocytes. They are intimately related with activation and suppression of the immune system and cell division. The interleukins are synthesized mostly by helper CD4⁺ T lymphocytes, monocytes, macrophages and endothelial cells.

There have been identified 40 interleukins so far and some of them are further divided into subtypes (e.g. IL-1α, IL-1β). These interleukins are grouped in families based on sequence homology and receptor chain similarities or functional properties.

The interleukin-1 family is composed by 11 cytokines: 7 ligands with agonist activity (IL-1α, IL-1β, IL-18, IL-33, IL-36α, IL-36β and IL-36γ), 3 receptor antagonists (IL-1Ra, IL-36Ra and IL-38) and 1 anti-inflammatory cytokine (IL-37). All members of the family except IL-18 and IL-33 have genes encoding on chromosome 2 in a 400 kb region in human species. Despite the fact that all the cytokines are extracellular, they are synthesized without a hydrophobic leader sequence and are not secreted via reticulum endoplasmic-Golgi pathway, with the exception of IL-1Rα. These cytokines bind to closely related receptors, and many of the encoding genes are clustered in a short region of chromosome 2. The receptors contain extracellular immunoglobulin domains and a toll/IL-1 receptor (TIR) domain in the cytoplasmic portion.

The interleukin-2 (IL-2) cytokine family, also known as the common γ-chain family, is composed by IL-2, -4, -7, -9, -15 and -21. All these interleukins bind to the common γc receptor, also called CD132. These cytokines act as growth and proliferation factors for progenitors and mature cells. IL-2 is mainly produced by CD4⁺ and CD8⁺ T cells, but can be also expressed by dendritic cells and NKs. The IL-2R is composed by three subunits (CD25, CD122 and common γc), all necessary to binding to IL-2. IL-2 acts in the development of regulatory T (Treg) cells, as a B cell growth factor, stimulates antibody synthesis and promotes proliferation and differentiation of NK cells and T helpers. IL-2 has been extensively used as an anti-cancer therapy.

In certain embodiments, the cytokine is IL-2, modified IL-2, IL-2 derivatives, IL-2 mutants or combinations thereof. In certain embodiments, the IL-2 is present in the cryopreservative medium in a low dose.

Other Cytokines

Chemokines represent a large family of structurally homologous cytokines that stimulate leukocytes movement and regulate the migration of them from the blood to tissues, in a process named chemotaxis. They control homeostatic immune cells, such as neutrophils, B cells, and monocytes, trafficking between the bone marrow, blood and peripheral tissues. Therefore, they can be classified as chemotactic cytokines.

There are about 50 human chemokines, classified into 4 families according to the location of N-terminal cysteine residues. The two major families are CC and CXC chemokines, in which the cysteine residues are adjacent on CC family, and are separated by one amino acid on CXC family. In general, members of CC chemokines are chemotactic for monocytes, and a small subset of lymphocytes, while CXC chemokines are more specific for neutrophils. The best-known chemokine is IL-8, or CXCL8, which belongs to the CXC chemokine family, and is responsible for neutrophil recruitment and for the maintenance of the inflammatory reaction. On the other hand, the monocyte chemoattractant protein-1 (MCP-1) or CCL2, and CCL11 or eotaxin, are examples of CC chemokines, which acts on recruitment of a variety of leukocytes, but especially monocytes, and eosinophils, respectively.

Besides chemokines, there are cytokines that stimulates hematopoiesis, such as the colony-stimulating factors (CSFs), which contributes to the growth of progenitors of monocytes, neutrophils, eosinophils and basophils, as well as activating macrophages. Immune and inflammatory reactions uses leukocytes, due to the recruitment induced by some kinds of cytokines. Additionally, the GM-CSF (granulocyte-macrophage colony-stimulating factor) and M-CSF (macrophage colony-stimulating factor) have, like some other cytokines, a pro-inflammatory action.

Other cytokines can be highlighted: TGF-β, LIF, Eta-1 and oncostatin M. The TGF-β is responsible for the chemoattraction of monocytes and macrophages, but also it has an anti-inflammatory effect, by inhibiting the lymphocyte proliferation. LIF and oncostatin M induce the production of acute-phase protein, while Eta-1 stimulates the production of IL-2, and inhibits the production of IL-10.

TABLE 1
Characteristics of selected cytokines

| Cytokines | Source |
|---|---|
| IL-1(IL-1α and β) | Macrophages, dendritic cells, endothelial cells, other cells |
| IL-2 | TH1 cells |
| IL-3 | TH, Tc and NK cells |
| IL-4 | TH2 cells, mast cells, NK cells |
| IL-5 | TH2 cells, mast cells |
| IL-6 | Macrophages, TH2 cells |
| IL-8 | Bone marrow, thymus |
| IL-9 | TH2 cells |
| IL-10 | TH2 cells |
| IL-11 | Bone marrow |
| IL-12 | Macrophages, B cells |
| IL-13 | TH cells |
| IL-16 | Tc cells |
| IL-18 | Hematopoietic and non-hematopoietic lineage cells |
| IFN-α | Leukocytes |
| IFN-β | Fibroblasts |
| IFN-γ | TH1, CTL, NK |
| TNF-α | Macrophages |
| TNF-β | T cells |
| Cytokines | Target |
| IL-1(IL-1α and β) | TH and B cells and various other tissues |
| IL-2 | TH, Tc and NK cells |
| IL-3 | Hematopoietic and mast cells |
| IL-4 | B cells, T cells, mast cells, macrophages |
| IL-5 | Eosinophils |
| IL-6 | Plasma cells, B cells and others |
| IL-8 | Neutrophils |
| IL-9 | TH cells, mast cells, eosinophils |
| IL-10 | Macrophages, APC |
| IL-11 | B-cell progenitors and others |
| IL-12 | Tc, NK and LAK cells |
| IL-13 | Macrophages, B cells |
| IL-16 | TH cells |
| IL-18 | T cells, NK cells |
| IFN-α | |
| IFN- β | |
| IFN-γ | Various cells including macrophages |
| TNF-α | Tumor cells, polymorphonuclear leukocytes, macrophages |
| TNF-β | Tumor cells, neutrophils, macrophages |
| Cytokines | Biological Role |
| IL-1(IL-1α and β) | Activation |
| IL-2 | T cell and NK proliferation and induction of activity |
| IL-3 | Progenitor cell proliferation and differentiation |
| IL-4 | Proliferation, isotype switching, induction of MHC class II expression |
| IL-5 | Proliferation and differentiation |
| IL-6 | Differentiation and antibody secretion |
| IL-8 | Chemoattractant |
| IL-9 | Induces inflammatory responses |
| IL-10 | Anti-inflammatory cytokine inhibits cytokine production |
| IL-11 | Differentiation |
| IL-12 | Proliferation and differentiation in synergy with IL-2 |
| IL-13 | Inhibition of inflammatory cytokines, regulation of inflammation. Parasitic infections |
| IL-16 | Chemotaxis |
| IL-18 | Proinflammatory cytokine; IFN-γ inducing factor |
| IFN-α | Inhibitor of viral replication |
| IFN-β | Inhibitor of viral replication |
| IFN-γ | Inhibitor of viral replication. Inhibitor of cell proliferation. |
| | Inhibitor of IL-4 induced isotype switching |
| TNF-α | Cytotoxicity, induction of cytokine secretion |
| TNF-β | Cytotoxicity, phagocytosis |

In certain embodiments, the cytokines are combined in a physiological solution, such as saline and dextrose, as well as biological media, e.g., Dulbecco's Modified Eagle Medium (DMEM), Dulbecco's Modified Eagle Medium: Nutrient Mixture F-12 (DMEM/F12), F10 Nutrient Mixture, Ham's F12 Nutrient Mixture, Media 199, Minimum Essential Media (MEM), RPMI Medium 1640 (RPMI-1640), Opti-MEM I Reduced Serum Media, Iscove's Modified Dulbecco's Medium (IMDM), Eagle's Minimal Essential Medium (EMEM), phosphate buffered saline (PBS), Hank's Balanced Salt Solution (HBSS), Plasma-Lyte, X-VIVO, 293 Media, CHO Media, DG44 Media, Cytogenetics Media, Grace's Insect Media, Carbenicillin High Five Media, Hybridoma Media, Neuronal Cell Media, Fosmidomycin, Virus Production Media, Stem Cell Differentiation Kits, Protein Expression (PEM) Media, Primary Cell Media, Schneider's Drosophila Media, Stem Cell Medium, T Cell Media, Sf9 and Sf21 Media, water, saline, dextrose, dextrose in water or saline pharmaceutically or biologically acceptable or relevant excipients (for example, an excipient should generally be non-toxic to the subject), sterile water, electrolytes such as sodium chloride, and combinations thereof.

In certain embodiments, the cytokines are combined with cryopreservative or preservative media that are commercially available. For example, cryopreservation media are available from Akron Biotech, Boca Raton Fla.; Sigma-Aldrich, St. Louis, Mo.; STEMCELL Technologies, Cambridge Mass.

In certain embodiments, the cryopreservative media comprise one or more therapeutic macromolecules. Therapeutic macromolecules can include therapeutic proteins or therapeutic polynucleotides. Therapeutic proteins include, but are not limited to, infusible therapeutic proteins, enzymes, enzyme cofactors, hormones, blood clotting factors, cytokines and interferons, growth factors, monoclonal antibodies, and polyclonal antibodies (e.g., that are administered to a subject as a replacement therapy), and proteins associated with Pompe's disease (e.g., acid glucosidase alpha, rhGAA (e.g., Myozyme and Lumizyme (Genzyme)). Therapeutic proteins also include proteins involved in the blood coagulation cascade. Therapeutic proteins include, but are not limited to, Factor VIII, Factor VII, Factor IX, Factor V, von Willebrand Factor, von Heldebrant Factor, tissue plasminogen activator, insulin, growth hormone, erythropoietin alpha, VEGF, thrombopoietin, lysozyme, antithrombin and the like. Therapeutic proteins also include adipokines, such as leptin and adiponectin.

Examples of therapeutic proteins used in enzyme replacement therapy of subjects having a lysosomal storage disorder include, but are not limited to, imiglucerase for the treatment of Gaucher's disease (e.g., CEREZYME®), α-galactosidase A (α-gal A) for the treatment of Fabry disease (e.g., agalsidase beta, FABRAYZYME®), acid α-glucosidase (GAA) for the treatment of Pompe disease (e.g., acid glucosidase alpha, LUMIZYME™, MYOZYME™), arylsulfatase B for the treatment of mucopolysaccharidoses (e.g., laronidase, ALDURAZYME™, idursulfase, ELAPRASE™, arylsulfatase B, NAGLAZYME™)), pegloticase (KRYSTEXXA™) and pegsiticase.

Examples of enzymes include oxidoreductases, transferases, hydrolases, lyases, isomerases, asparaginases, uricases, glycosidases, asparaginases, uricases, proteases, nucleases, collagenases, hyaluronidases, heparinases, heparanases, lysins, and ligases.

Therapeutic proteins may also include any enzyme, toxin, or other protein or peptide isolated or derived from a bacterial, fungal, or viral source.

Examples of hormones include Melatonin (N-acetyl-5-methoxytryptamine), Serotonin, Thyroxine (or tetraiodothyronine) (a thyroid hormone), Triiodothyronine (a thyroid hormone), Epinephrine (or adrenaline), Norepinephrine (or noradrenaline), Dopamine (or prolactin inhibiting hormone), Antimullerian hormone (or mullerian inhibiting factor or hormone), Adiponectin, Adrenocorticotropic hormone (or corticotropin), Angiotensinogen and angiotensin, Antidiuretic hormone (or vasopressin, arginine vasopressin), Atrial-natriuretic peptide (or atriopeptin), Calcitonin, Cholecystokinin, Corticotropin-releasing hormone, Erythropoietin, Follicle-stimulating hormone, Gastrin, Ghrelin, Glucagon, Glucagon-like peptide (GLP-1), GIP, Gonadotropin-releasing hormone, Growth hormone-releasing hormone, Human chorionic gonadotropin, Human placental lactogen, Growth hormone, Inhibin, Insulin, Insulin-like growth factor (or somatomedin), Leptin, Luteinizing hormone, Melanocyte stimulating hormone, Orexin, Oxytocin, Parathyroid hormone, Prolactin, Relaxin, Secretin, Somatostatin, Thrombopoietin, Thyroid-stimulating hormone (or thyrotropin), Thyrotropin-releasing hormone, Cortisol, Aldosterone, Testosterone, Dehydroepiandrosterone, Androstenedione, Dihydrotestosterone, Estradiol, Estrone, Estriol, Progesterone, Calcitriol (1,25-dihydroxyvitamin D3), Calcidiol (25-hydroxyvitamin D3), Prostaglandins, Leukotrienes, Prostacyclin, Thromboxane, Prolactin releasing hormone, Lipotropin, Brain natriuretic peptide, Neuropeptide Y, Histamine, Endothelin, Pancreatic polypeptide, Renin, and Enkephalin.

Examples of blood or blood coagulation factors include Factor I (fibrinogen), Factor II (prothrombin), tissue factor, Factor V (proaccelerin, labile factor), Factor VII (stable factor, proconvertin), Factor VIII (antihemophilic globulin), Factor IX (Christmas factor or plasma thromboplastin component), Factor X (Stuart-Prower factor), Factor Xa, Factor XI, Factor XII (Hageman factor), Factor XIII (fibrin-stabilizing factor), von Willebrand factor, prekallikrein (Fletcher factor), high-molecular weight kininogen (HMWK) (Fitzgerald factor), fibronectin, fibrin, thrombin, antithrombin III, heparin cofactor II, protein C, protein S, protein Z, protein Z-related protease inhibitor (ZPI), plasminogen, alpha 2-antiplasmin, tissue plasminogen activator (tPA), urokinase, plasminogen activator inhibitor-1 (PAI1), plasminogen activator inhibitor-2 (PAI2), cancer procoagulant, and epoetin alpha (Epogen, Procrit).

Examples of growth factors include Adrenomedullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha (TGF-α), Transforming growth factor beta (TGFβ), Tumor necrosis factor-alpha (TNFα), Vascular endothelial growth factor (VEGF), Wnt Signaling Pathway, placental growth factor (PlGF) and (Fetal Bovine Somatotrophin) (FBS).

Examples of monoclonal antibodies include Abagovomab, Abciximab, Adalimumab, Adecatumumab, Afelimomab, Afutuzumab, Alacizumab pegol, ALD, Alemtuzumab, Altumomab pentetate, Anatumomab mafenatox, Anrukinzumab, Anti-thymocyte globin, Apolizumab, Arcitumomab, Aselizumab, Atlizumab (tocilizumab), Atorolimumab, Bapineuzumab, Basiliximab, Bavituximab, Bectumomab, Belimumab, Benralizumab, Bertilimumab, Besilesomab, Bevacizumab, Biciromab, Bivatuzumab mertansine, Blinatumomab, Brentuximabvedotin, Briakinumab, Canakinumab, Cantuzumab mertansine, Capromabpendetide, Catumaxomab, Cedelizumab, Certolizumab pegol, Cetuximab, Citatuzumab bogatox, Cixutumumab, Clenoliximab, Clivatuzumab tetraxetan, Conatumumab, Dacetuzumab, Daclizumab, Daratumumab, Denosumab, Detumomab, Dorlimomab aritox, Dorlixizumab, Ecromeximab, Eculizumab, Edobacomab, Edrecolomab, Efalizumab, Efungumab, Elotuzumab, Elsilimomab, Enlimomabpegol, Epitumomab cituxetan, Epratuzumab, Erlizumab, Ertumaxomab, Etaracizumab, Exbivirumab, Fanolesomab, Faralimomab, Farletuzumab, Felvizumab, Fezakinumab, Figitumumab, Fontolizumab, Foravirumab, Fresolimumab, Galiximab, Gantenerumab, Gavilimomab, Gemtuzumabozogamicin, GC1008, Girentuximab, Glembatumumab vedotin, Golimumab, Gomiliximab, Ibalizumab, Ibritumomab tiuxetan, Igovomab, Imciromab, Infliximab, Intetumumab, Inolimomab, Inotuzumab ozogamicin, Ipilimumab, Iratumumab, Keliximab, Labetuzumab, Lebrikizumab, Lemalesomab, Lerdelimumab, Lexatumumab, Libivirumab, Lintuzumab, Lorvotuzumab mertansine, Lucatumumab, Lumiliximab, Mapatumumab, Maslimomab, Matuzumab, Mepolizumab, Metelimumab, Milatuzumab, Minretumomab, Mitumomab, Morolimumab, Motavizumab, Muromonab-CD3, Nacolomab tafenatox, Naptumomab estafenatox, Natalizumab, Nebacumab, Necitumumab, Nerelimomab, Nimotuzumab, Nofetumomab merpentan, Ocrelizumab, Odulimomab, Ofatumumab, Olaratumab, Omalizumab, Oportuzumab monatox, Oregovomab, Otelixizumab, Pagibaximab, Palivizumab, Panitumumab, Panobacumab, Pascolizumab, Pemtumomab, Pertuzumab, Pexelizumab, Pintumomab, Priliximab, Pritumumab, Rafivirumab, Ramucirumab, Ranibizumab, Raxibacumab, Regavirumab, Reslizumab, Rilotumumab, Rituximab, Robatumumab, Rontalizumab, Rovelizumab, Ruplizumab, Satumomab pendetide, Sevirumab, Sibrotuzumab, Sifalimumab, Siltuximab, Siplizumab, Solanezumab, Sonepcizumab, Sontuzumab, Stamulumab, Sulesomab, Tacatuzumab tetraxetan, Tadocizumab, Talizumab, Tanezumab, Taplitumomab paptox, Tefibazumab, Telimomab aritox, Tenatumomab, Teneliximab, Teplizumab, Ticilimumab (tremelimumab), Tigatuzumab, Tocilizumab (atlizumab), Toralizumab, Tositumomab, Trastuzumab, Tremelimumab, Tucotuzumab celmoleukin, Tuvirumab, Urtoxazumab, Ustekinumab, Vapaliximab, Vedolizumab, Veltuzumab, Vepalimomab, Visilizumab, Volociximab, Votumumab, Zalutumumab, Zanolimumab, Ziralimumab, and Zolimomab aritox. Monoclonal antibodies further include anti-TNFα antibodies.

Examples of infusion therapy or injectable therapeutic proteins include, for example, Tocilizumab (Roche/ACTEMRA®), alpha-1 antitrypsin (Kamada/AAT), HEMATIDE® (Affymax and Takeda, synthetic peptide), albinterferon alpha-2b (Novartis/ZALBIN™) RHUCIN® (Pharming Group, C1 inhibitor replacement therapy), tesamorelin (Theratechnologies/EGRIFTA®, synthetic growth hormone-releasing factor), ocrelizumab (Genentech, Roche and Biogen), belimumab (GlaxoSmithKline/BENLYSTA®), pegloticase (Savient Pharmaceuticals/KRYSTEXXA™), pegsiticase, taliglucerase alpha (Protalix/Uplyso), agalsidase alpha (Shire/REPLAGAL®), velaglucerase alpha (Shire), and Keyhole Limpet Hemocyanin (KLH).

Additional therapeutic proteins include, for example, engineered proteins, such as Fc fusion proteins, bispecific antibodies, multi-specific antibodies, nanobodies, antigen-binding proteins, antibody fragments, and protein conjugates, such as antibody drug conjugates.

Therapeutic polynucleotides include, but are not limited to nucleic acid aptamers such as Pegaptanib (Macugen, a pegylated anti-VEGF aptamer), antisense therapeutics such as antisense poly- or oligonucleotides (e.g., antiviral drug Fomivirsen, or Mipomersen, an antisense therapeutic that targets the messenger RNA for apolipoprotein B for reduction of cholesterol level); small interfering RNAs (siRNAs) (e.g., dicer substrate siRNA molecules (DsiRNAs) which are 25-30 base pair asymmetric double-stranded RNAs that mediate RNAi with extremely high potency); or modified messenger RNAs (mmRNAs) such as those disclosed in U.S. Pat. No. 9,428,535 to de Fougerolles et al. and in U.S. Pat. No. 8,710,200 to Schrum et al.

Additional therapeutic macromolecules useful in accordance with aspects of this invention will be apparent to those of skill in the art, and the invention is not limited in this respect.

In certain embodiments, a preservative or cryopreservative composition comprises a polyamino acid, an organic amphoteric agent, a saccharide, at least one cytokine or combinations thereof.

In certain embodiments, the organic amphoteric agent is ectoine or derivatives thereof. In certain embodiments, a derivative of ectoine comprises: acetylhydroxectoine, hydroxyectoine, homoectoin, stearoylhydroxyectoine, myristylectoin, or combinations thereof. In certain embodiments, the polyamino acid is poly-L-lysine.

In certain embodiments, the saccharide comprises: a monosaccharide, disaccharide, oligosaccharide, polysaccharide or combinations thereof. In certain embodiments, the saccharide comprises: dextran, α-D-glucopyranosyl-(1→1)-α-D-glucopyranoside (trehalose) or a combination thereof.

In certain embodiments the preservative or cryopreservative composition further comprises one or more other compounds comprising: polysucrose, polyamines, DHMICA (4,5-dihydro-2-methylimidazole-4-carboxylate), cDPG (cyclic 2,3-diphosphoglycerate, potassium salt), DGP (diglycerol phosphate), polyethylene glycol (PEG), glycoin, firoin, succinic anhydride, sodium hydroxide or combinations thereof. In certain embodiments, the compound is polysucrose.

In other embodiments, a preservative or cryopreservative composition comprises ectoine or derivatives thereof, a polyamino acid, a saccharide, at least one cytokine or combinations thereof. In certain embodiments, the cryopreservative composition further comprises one or more of: polysucrose, DHMICA (4,5-dihydro-2-methylimidazole-4-carboxylate), cDPG (cyclic 2,3-diphosphoglycerate, potassium salt), DGP (diglycerol phosphate), polyethylene glycol (PEG), glycoin, firoin, succinic anhydride, sodium hydroxide or combinations thereof.

In another embodiment, the preservative or cryopreservative composition comprises: poly-L-lysine, ectoine or derivatives thereof, dextran, at least one cytokine or combinations thereof. In certain embodiments, the preservative or cryopreservative composition comprises: ectoine or derivatives thereof, poly-L-lysine, trehalose, at least one cytokine or combinations thereof. In certain embodiments, the preservative or cryopreservative composition comprises: ectoine or derivatives thereof, poly-L-lysine, dextran or combinations thereof. In certain embodiments, the preservative or cryopreservative composition comprises: ectoine or derivatives thereof, poly-L-lysine, dextran, polysucrose, at least one cytokine or combinations thereof. In certain embodiments, the preservative or cryopreservative composition comprises: ectoine or derivatives thereof, poly-L-lysine, trehalose, polysucrose, at least one cytokine or combinations thereof. In certain embodiments, the preservative or cryopreservative composition comprises: ectoine or derivatives thereof, poly-L-lysine, dextran, trehalose, polysucrose, at least one cytokine or combinations thereof.

In another embodiment, a composition comprises poly-L-lysine, succinic anhydride, a hydroxide, cell-culture medium, at least one cytokine or combinations thereof. In certain embodiments, the composition further comprises: ectoine or derivatives thereof, trehalose or combinations thereof. In certain embodiments, the preservative or cryopreservative composition comprises: ectoine or derivatives thereof, dextran, at least one cytokine or combinations thereof. In certain embodiments, the preservative or cryopreservative composition comprises: ectoine or derivatives thereof, dextran, polysucrose, at least one cytokine or combinations thereof. In certain embodiments, the preservative or cryopreservative composition comprises: ectoine or derivatives thereof, trehalose, polysucrose, at least one cytokine or combinations thereof. In certain embodiments, the preservative or cryopreservative composition comprises: ectoine or derivatives thereof, dextran, trehalose, polysucrose, at least one cytokine or combinations thereof.

In another embodiment, a composition comprises a carboxylated-polyamino acid, ectoine or derivatives thereof, a polysaccharide, at least one cytokine or combinations thereof. The carboxylated polyamino acid may be derived from a polylysine. Polylysine is intended to include ε-poly-L-lysine or ε-poly-D-lysine or α-poly-L-lysine. The polylysine may include an average molecular weight of about 1,000-20,000 Daltons, and particularly between about 1,000-10,000 Daltons. In certain embodiments, the saccharide comprises: dextran, α-D-glucopyranosyl-(1→1)-α-D-glucopyranoside (trehalose) or a combination thereof. In certain embodiments, the composition further comprises polysucrose, polyamines, DHMICA (4,5-dihydro-2-methylimidazole-4-carboxylate), cDPG (cyclic 2,3-diphosphoglycerate, potassium salt), DGP (diglycerol phosphate), polyethylene glycol (PEG), glycoin, firoin, succinic anhydride, sodium hydroxide or combinations thereof.

In yet another embodiment, a composition comprises at least one cytokine, a polyamino acid, ectoine or derivatives thereof, dextran, α-D-glucopyranosyl-(1→1)-α-D-glucopyranoside (trehalose) polysucrose, polyamines, DHMICA (4,5-dihydro-2-methylimidazole-4-carboxylate), cDPG (cyclic 2,3-diphosphoglycerate, potassium salt), DGP (diglycerol phosphate), polyethylene glycol (PEG), glycoin, firoin, succinic anhydride, sodium hydroxide or combinations thereof.

In yet another embodiment, a composition comprises at least one cytokine, ectoin, hydroxyectoin, glycoin, firoin, firoin-A, cDPG (cyclic 2,3-diphosphoglycerate, potassium salt), DGP (diglycerol phosphate), DIP (di-myo-inositolphosphate), homoectoin, DHMICA (4,5-dihydro-2-methylimidazole-4-carboxylate), acetyl-hydroxyectoin, myristylectoin, stearoylhydroxyectoin, or combinations thereof. These can be combined with one or more other compounds such as dextran, trehalose, polysucrose or combinations thereof.

In some embodiments, the preservation or cryopreservation media comprise at least one cytokine, a pharmaceutical excipient or a combination thereof, wherein the pharmaceutically acceptable excipient is at least 95%, 96%, 97%, 98%, 99%, or 100% pure. In some embodiments, the excipient is approved for use in humans and for veterinary use. In some embodiments, the excipient is approved for use in humans by the United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

In some embodiments, the preservative or cryopreservative compositions further comprise at least one cytokine, an aldose, a ketose, an amino sugar, a saccharide (e.g., a disaccharide, a polysaccharide, etc.), or combinations thereof. In some embodiments, the preservative or cryopreservative compositions comprise at least one cytokine sucrose, dextrose, glucose, lactose, trehalose, dextran e.g. dextran-40, arabinose, pentose, ribose, xylose, galactose, hexose, idose, monnose, mannose, talose, heptose, fructose, gluconicacid, sorbitol, mannitol, methyl α-glucopyranoside, maltose, isoascorbic acid, ascorbic acid, lactone, sorbose, glucaric acid, erythrose, threose, arabinose, allose, altrose, gulose, erythrulose, ribulose, xylulose, psicose, tagatose, glucuronicacid, gluconic acid, glucaric acid, galacturonic acid, mannuronic acid, glucosamine, galactosamine, neuraminic acid, arabinans, fructans, fucans, galactans, galacturonans, glucans, mannans, xylans, levan, fucoidan, carrageenan, galactocarolose, pectins, pectic acids, amylose, pullulan, glycogen, amylopectin, cellulose, dextran, pustulan, chitin, agarose, keratin, chondroitin, dermatan, hyaluronic acid, alginic acid, xanthin gum, starch, polyethyleneglycol, dimethyl sulfoxide, ethylene glycol, propylene glycol, propylene, glycol, polyvinvyl pyrrolidone, glycerol, polyethylene oxide, polyether, serum, or combinations thereof.

In certain embodiment, a preservative or cryoprotective composition comprises at least one cytokine and one or more cryoprotective agents. In preferred embodiments, the preservative or cryoprotective agent is non-toxic to the cellular matter under the conditions at which it is used (e.g. at a particular concentration, for a particular exposure time and to cells in a medium of a particular osmolality). A preservative or cryoprotective agent may be cell permeating or non-permeating. Examples of preservative or cryoprotective agents include but are not limited to, dehydrating agents, osmotic agents and vitrification solutes (i.e., solutes that aid in the transformation of a solution to a glass rather than a crystalline solid when exposed to low temperatures). In some embodiments, a preservative or cryoprotective agent can be a naturally-occurring cryoprotective agent such as ectoin and/or hydroxyectoin. Other examples of naturally occurring agents or cryoprotectants include, without limitation, anti-freeze proteins, saccharides, ice nucleating agents, compatible solutes, sugars, polyols, glucose, sucrose, glycerol and the like. These can be isolated from nature, synthesized in the laboratory, or obtained from commercial sources. Natural sources include insects, fish, amphibians, animals, birds and plants. Most notably, Arctic and Antarctic insects, fish and amphibians.

In certain embodiments, the preservation or cryopreservation media compositions further include one or more therapeutic agents, hormones, growth factors, lipids, cytokines, oligonucleotides, polynucleotides, proteins, polypeptides, peptides, small molecules, chemotherapeutic agents and the like (e.g., polyphenols, fatty alcohols).

Cell Culture Media: The cell culture media include, but are not limited to, a basal cell culture medium selected from the group consisting of basal medium eagle (BME), Eagle's minimum essential medium (MEM or Eagle's MEM), Earle's balanced salt solution (EBSS), Dulbecco's modified Eagle's medium (DMEM), HAM's F-10 medium, HAM's F-12 medium, DMEM-F12 medium, Roswell Park Memorial Institute 1640 (RPMI 1640 or RPMI), Leibowitz's medium (L-15-medium), combinations thereof or modified versions thereof. In some examples, the cell culture medium as described herein may include Iscove's Modified Dulbecco's Medium (IMDM); IMDM with HEPES and L-Glutamine; IMDM with HEPES and without L-Glutamine; RPMI 1640 with L-Glutamine; RPMI 1640 with HEPES, L-Glutamine and/or Penicillin-Streptomycin; Minimal Essential Medium-alpha (MEM-alpha); DMEM:F12 1:1 with L-Glutamine; DME/F12; Basal Medium Eagle with Earle's BSS; GMEM (Glasgow's MEM); GMEM with L-glutamine; F-10; F-12; Ham's F-10 with L-Glutamine; Ham's F-12 with L-Glutamine; L-15 (Leibovitz) (2×) without L-Glutamine or Phenol Red; L-15 (Leibovitz) without L-Glutamine; McCoy's 5A Modified Medium; Medium 199; MEM Eagle without L-Glutamine or Phenol Red (2×); MEM Eagle-Earle's BSS with L-glutamine; MEM Eagle-Earle's BSS without L-Glutamine; MEM Eagle-Hanks BSS without L-Glutamine; Hank's Balanced Salt Solution (HBSS); Plasma-Lyte; NCTC-109 with L-Glutamine; Richter's CM Medium with L-Glutamine; physiological solutions; ophthalmic solutions; and hydrolysate-containing media.

The cell culture media are aqueous-based (but can be reconstituted from dry powder and/or frozen components), comprising a number of ingredients in water, liquid, and/or an aqueous solution. The term “component” refers to any compound, whether of chemical or biological origin, that can be used in cell culture media to maintain or promote the growth of proliferation of cells. The terms “component,” “nutrient” and ingredient” can be used interchangeably and are all meant to refer to such compounds. Typical ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

In some embodiments, the medium is substantially free of one or more specified components. In certain embodiments, “substantially free” refers to a low amount of the component that has no statistically significant effect on cell growth and/or differentiation state. In some embodiments, “substantially free” refers to less than 1%, 0.1%, 0.01%, 0.001%, or 0.0001% v/v of a liquid or w/v of a solute. In some embodiments, “substantially free” refers to a concentration of less than 0.001, 0.0001, 0.00001, 0.000001, or 0.0000001 mg/L. In some embodiments, “substantially free” refers to a concentration of less than 10 nM, 1 nM, 100 pM, 10 pM, or 1 pM.

In certain embodiments, a culture medium comprises one or more cytokines, one or more antioxidants; nucleotide synthesis and salvage pathway precursors; lipid synthesis precursors; agonists of intracellular cAMP level; hormones and growth factors; and serum. The media may additionally comprise other components such as amino acid supplements, vitamins necessary for cell growth/proliferation, trace minerals, inorganic salts, energy sources (e.g. for glycolysis), and other components such as pH indicators, etc. In other words, ingredients may include amino acids, vitamins, inorganic salts, adenine, D-glucose, N-[2-hydroxyethyl]piperazine-N′-[2-ethanesulfonic acid] (HEPES), hydrocortisone, insulin, lipoic acid, phenol red, phosphoethanolamine, putrescine, sodium pyruvate, triiodothyronine (T3), thymidine and transferrin. Each of these ingredients may be obtained commercially, for example from Sigma (Saint Louis, Mo.).

The antioxidants may include, without limitation, one or more of the following: beta-carotene, vitamin E, vitamin C (ascorbic acid), vitamin K3, glutathione (reduced), niacin (or niacinamide), or DTT (dithiothreitol). The antioxidants may optionally be supplemented with trace metals, including Zn, Se, Cr, Cu, Mg, or Mn.

In certain embodiments, trace minerals are included in the cell culture media. For example, glutathione peroxidase uses selenium and glutathione superoxide uses copper as a cofactor.

Vitamin ingredients which may be included in the media include biotin, choline chloride, D-Ca⁺⁺-pantothenate, folic acid, i-inositol, niacinamide, pyridoxine, riboflavin, thiamine and vitamins A and B12. These vitamins may be obtained commercially, for example from Sigma (Saint Louis, Mo.).

Protein synthesis precursors include amino acid ingredients. In one embodiment, the amino acid ingredients which may be included in the media include L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine and L-valine. These amino acids may be obtained commercially, for example from Sigma (Saint Louis, Mo.).

Alternatively, in some other embodiments, only essential amino acids are included in the media. Certain cells, such as human cells must have adequate amounts of 9 amino acids to survive. These so called “essential” amino acids cannot be synthesized from other precursors. However, cysteine can partially meet the need for methionine (they both contain sulfur), and tyrosine can partially substitute for phenylalanine. Such essential amino acids include: Histidine, Isoleucine, Leucine, Lysine, Methionine (and/or cysteine), Phenylalanine (and/or tyrosine), Threonine, Tryptophan, and Valine. In certain embodiments, only Histidine, Isoleucine, Leucine, Lysine, Threonine, Tryptophan, and Valine are included.

In certain embodiments, the media may also include one or more cytokines, hormones, such as: progesterone, testosterone, hydrocortisone, or estrogen, and/or one or more growth factors such as: insulin and EGF (epidermal growth factor). The media may also include lipid synthesis precursors, such as: cholesterol, linoleic acid, lipoic acid, or O-phosphoryl ethanolamine. The media may also include one or more cAMP agonists or agents that increase intracellular cAMP levels. Included are agents which induce a direct increase in intracellular cAMP levels (e.g., dibutyryl cAMP), agents which cause an increase in intracellular cAMP levels by an interaction with a cellular G-protein (e.g., cholera toxin and forskolin), agents which cause an increase in intracellular cAMP levels by acting as agonists of B-adrenergic receptors (e.g., isoproterenol) and agents which cause an increase in intracellular cAMP levels by inhibiting the activities of cAMP phosphodiesterases (e.g., isobutylmethylxanthine (IBMX) and theophylline). These cAMP-increasing agents are available commercially, e.g. from Sigma (St. Louis, Mo.), and are used at concentrations approximating those described in Green (Proc. Natl. Acad. Sci. USA 15:801-811 (1978)). The subject media may also comprise one or more carbohydrate synthesis and energy metabolism precursors, such as D-glucose, sodium pyruvate, etc. The subject media may also comprise one or more carrier proteins, such as bovine serum albumin (BSA). Carrier protein may be a protein which transports specific substances through the cell membrane in which it is embedded and into the cell. Different carrier proteins may be required to transport different substances, as each one is designed to recognize only one substance, or group of similar substances. Certain carrier proteins may bind to one or more media components (such as growth factors, etc.) and confer them extra stability in the media, or to facilitate certain biological processes (e.g. acyl-carrier protein, sterol carrier protein, hormone carrier protein, etc.). The subject media may also comprise one or more surfactants, such as nonionic surfactants Tween 60 or Tween 80. Although not considered essential, the subject media may additionally comprise one or more buffer systems, such as HEPES and sodium bicarbonate buffer systems, such that a balanced pH is maintained in long-term culture. Frequent, constant or continuous change of culture medium may also help to restore medium pH in fast growing cells.

As will be readily apparent to one of ordinary skill in the art, the concentration of a given ingredient can be increased or decreased beyond the range disclosed and the effect of the increased or decreased concentration can be determined using only routine experimentation. The optimization of the present media formulations for any specific cell type can be carried out using approaches described by Ham (Ham, Methods for Preparation of Media, Supplements and Substrata for Serum-Free Animal Culture, Alan R. Liss, Inc., New York, pp. 3-21, 1984) and Waymouth (Waymouth, C., Methods for Preparation of Media, Supplements and Substrata for Serum-Free Animal Culture, Alan R. Liss, Inc., New York, pp. 23-68, 1984). The optimal final concentrations for medium ingredients are typically identified either by empirical studies, in single component titration studies, or by interpretation of historical and current scientific literature. In single component titration studies, using animal cells, the concentration of a single medium component is varied while all other constituents and variables are kept constant and the effect of the single component on viability, growth or continued health of the animal cells is measured.

It will be understood that the cytokines embodied herein can exist in different forms, as known in the art (e.g., different naturally occurring or non-naturally occurring forms), and can be used as substitutes for one another. It will also be appreciated that where the instant application discloses cytokine, the invention should be understood to encompass embodiments in which any form of such cytokine having similar biological activity (or compound(s) that can be modified or metabolized in cell culture medium or intracellularly to provide a biologically active form) is used in the inventive media and/or method(s).

The medium ingredients can be dissolved in a liquid carrier or maintained in dry form. If dissolved in a liquid carrier the pH of the medium should be adjusted to about 7.0-7.6, The osmolarity of the medium should also be adjusted to about 275-350 mOsm. The type of liquid carrier and the method used to dissolve the ingredients into solution vary and can be determined by one of ordinary skill in the art with no more than routine experimentation. Typically, the medium ingredients can be added in any order.

The media are typically sterilized to prevent unwanted contamination. Sterilization may be accomplished, for example, by filtration through a low protein-binding membrane filter of about 0.1-1.0 μm pore size (available commercially, for example, from Millipore, Bedford, Mass.) after admixing the concentrated ingredients to produce a sterile culture medium. Alternatively, concentrated subgroups of ingredients may be filter-sterilized and stored as sterile solutions. These sterile concentrates can then be mixed under aseptic conditions with a sterile diluent to produce a concentrated 1× sterile medium formulation. Autoclaving or other elevated temperature-based methods of sterilization are not favored, since many of the components of the present culture media are heat labile and will be irreversibly degraded by temperatures such as those achieved during most heat sterilization methods.

Many tissue culture media typically contain one or more antibiotics, which are not necessary for cell growth/proliferation per se but are present to inhibit the growth of undesirable microbes, such as bacteria and/or fungi.

Antibiotics are natural chemical substances of relatively low molecular weight produced by various species of microorganisms, such as bacteria (including Bacillus species), actinomycetes (including Streptomyces) and fungi, that inhibit growth of or destroy other microorganisms. Substances of similar structure and mode of action may be synthesized chemically, or natural compounds may be modified to produce semi-synthetic antibiotics. These biosynthetic and semi-synthetic derivatives are also effective as antibiotics. The major classes of antibiotics are: (1) the β-lactams, including the penicillins, cephalosporins and monobactams; (2) the aminoglycosides, e.g., gentamicin, tobramycin, netilmycin, and amikacin; (3) the tetracyclines; (4) the sulfonamides and trimethoprim; (5) the fluoroquinolones, e.g., ciprofloxacin, norfloxacin, and ofloxacin; (6) vancomycin; (7) the macrolides, which include for example, erythromycin, azithromycin, and clarithromycin; and (8) other antibiotics, e.g., the polymyxins, chloramphenicol and the lincosamides.

### Cytokines

Cytokines are small secreted or membrane-bound proteins which play a crucial role in intercellular communication. Cytokine binding to its cognate receptor complex triggers a cascade of intracellular signaling events that enables the cell to sense and respond to its surroundings according to the needs of the cell, tissue and organ of which it is part of They are characteristically pleiotropic, meaning that they provoke a broad range of responses depending on the nature and the developmental state of the target cell. Moreover, some of them are highly redundant as several cytokines have overlapping activities, which enable them to functionally compensate for mutual loss. Cytokine activities can be autocrine, paracrine or endocrine causing a faint boundary between the designated term cytokine, peptide hormone and growth factor.

Cytokines resemble hormones in their biological activity and systemic level, for example, inflammation, systemic inflammatory response syndrome, acute phase reaction, wound healing, and the neuroimmune network.

Six different structural classes of cytokines are known: the α-helical bundle cytokines which comprises most interleukins, colony stimulating factors and hormones like growth hormone and leptin, the trimeric tumor necrosis factor (TNF) family, the cysteine knot growth factors, the β-trefoil fold group that includes the interleukin-1 family, the interleukin 17 (IL-17) family, and the chemokines.

Several cytokines have found important clinical applications. Examples include erythropoietin (EPO), granulocyte colony-stimulating factor (G-CSF), interferons α2 and -β and growth hormone. Conversely, often as a consequence of their pro-inflammatory nature, antagonizing selected cytokines also finds specific medical applications. Prime examples here are the strategies to block TNFα activity to combat autoimmune diseases such as rheumatoid arthritis. Because of these successes, strategies to optimize cytokine activities in the clinic are being explored. These include optimized half-life, reduced immunogenicity, targeted delivery to specific cell types and genetic fusions of two cytokines, so-called fusokines.

Comparison of cytokine sequences shows that primate cytokines (non-human) are closely related. An example: IL-1 alpha (IL-1α), IL-1 beta (IL-1β), IL-2, IL-4, IL-5, IL-6, IL-8, IL-10, IL-12, IL-15, IFN-alpha (IFN-α), IFN-gamma (IFN-Gγ), and TNF-alpha (TNF-α) which share 93% to 99% homology at the protein and nucleic acid level with the human sequences.

Cytokines can also be classified into family groups according to the types of secondary and tertiary structure. An example: IL-6, IL-11, ciliary neurotrophic factor (CNTF), leukemia inhibiting factor (LIF), OSM (Oncostatin-M), EPO (Erythropoietin), granulocyte colony stimulating factor (G-CSF), GH (Growth Hormone), PRL (Prolactin), IL-10, IFN-α, IFN-β form long chain 4 helix bundles. IL-2, IL-4, IL-7, IL-9, IL-13, IL-3, IL-5, GM-CSF, macrophage colony stimulating factor (M-CSF), stem cell factor (SCF), IFN-γ form short chain 4 helix bundles. Beta-trefoil structures are formed by IL-1α, IL-1β, acidic fibroblast growth factor (aFGF), basic FGF (bFGF), INT-2 (FGF3), FGF7. EGF, TGF-alpha (TGF-α), Betacellulin (BTC), SCDGF, Amphiregulin, HB-EGF, form EGF-like antiparallel beta-sheets.

Membrane-bound cytokines have been are associated with the extracellular matrix. The switching between soluble and membrane forms of cytokines is an important regulatory event. In some cases membrane forms of a cytokine have been found to be indispensable for normal development, with soluble forms being unable to entirely substitute for them.

Numerous cytokines are not stored inside cells though TGF-beta (TGF-β) and platelet derived growth factor (PDGF) are stored in platelets or TNF-α and IL-8 are found in human skin mast cells. Most of the cytokine's expression is regulated tightly at practically all levels. The factors are usually produced only by cells after cell activation in response to an induction signal. The production and secretion of cytokines and growth factors frequently is context dependent, i.e., their expression is influenced by individual signals received but also by the balance of signals received through one or more receptors (which themselves may be subject to inducible/repressible expression).

Cytokines can be broadly grouped based on whether they act on cells of the adaptive immune response or promote or inhibit inflammation. Further, they can be classified based on the receptors used for signaling. The tables below show a listing of cytokines and various families. These cytokines are exemplary and are included in the various embodiments of the invention either singular or as combinations of two or more.

TABLE 2
Classification of cytokines by immune response.

| | Family | Members |
|---|---|---|
| Adaptive immunity | Common γ chain | IL-2, IL-4, IL-7, IL-9, IL-15, IL-21 |
| | receptor ligands | |
| | Common β chain | IL-3, IL-5, GM-CSF |
| | (CD131) receptor ligands | |
| | Shared IL-2β chain | IL-2, IL-15 |
| | (CD122) | |
| | Shared receptors | IL-13 (IL-13R-IL-4R complex) |
| | | TSLP (TSLPR-IL-7R complex) |
| Pro-inflammatory | IL-1 | IL-1α, IL-1β, IL-1ra, IL-18, IL-33, IL-36α, |
| signaling | | IL-36β, IL-36γ, IL-36Ra, IL-37 and IL-1Hy2 |
| | IL-6 | IL-6, IL-11, IL-31, CNTF, CT-1, LIF, OPN, |
| | | OSM |
| | TNFα | TNFα, TNFβ, BAFF, APRIL |
| | IL-17 | IL-17A-F, IL-25 (IL-17E) |
| | Type I IFN | IFNα, IFNβ, IFNω, IFNκ, Limitin |
| | Type II IFN | IFNγ |
| | Type III IFN | IFNλ1 (IL-29), IFNλ2 (IL-28A), IFNλ3 |
| | | (IL-28B) |
| Anti-inflammatory | IL-12 | IL-12, IL-23, IL-27, IL-35 |
| signaling | IL-10 | IL-10, IL-19, IL-20, IL-22, IL-24, IL-26, IL-28, |
| | | IL-29 |

Abbreviations: CNTF, ciliary neurotrophic factor; CT-1, cardiotrophin-1; GM-CSF, granulocyte macrophage-colony stimulating factor; IFN, interferon; LIF, leukemia inhibitory factor; OPN, osteopontin, OSM, oncostatin M; TNFα tumor necrosis factor α; TSLP, thymic stromal lymphopoietin.

TABLE 3
Functions of cytokines, Cytokine action, defined by target cell and primary function.

| | Cytokine | Main sources | Target cell | Major function |
|---|---|---|---|---|
| Interleukins | IL-1 | Macrophages, B | B cells, NK | Pyrogenic, pro-inflammatory, |
| | | cells, DCs | cells, T-cells | proliferation and |
| | | | | differentiation, BM cell |
| | | | | proliferation |
| | IL-2 | T cells | Activated T | Proliferation and activation |
| | | | and B cells, NK | |
| | | | cells | |
| | IL-3 | T cells, NK cells | Stem cells | Hematopoietic precursor |
| | | | | proliferation and |
| | | | | differentiation |
| | IL-4 | Th cells | B cells, T cells, | Proliferation of B and |
| | | | macrophages | cytotoxic T cells, enhances |
| | | | | MHC class II expression, |
| | | | | stimulates IgG and IgE |
| | | | | production |
| | IL-5 | Th cells | Eosinophils, | Proliferation and maturation, |
| | | | B-cells | stimulates IgA and IgM |
| | | | | production |
| | IL-6 | Th cells, | Activated | Differentiation into plasma |
| | | macrophages, | B-cells, plasma | cells, IgG production |
| | | fibroblasts | cells | |
| | IL-7 | BM stromal | Stem cells | B and T cell growth factor |
| | | cells, epithelial | | |
| | | cells | | |
| | IL-8 | Macrophages | Neutrophils | Chemotaxis, pro-inflammatory |
| | IL-9 | T cell | T cell | Growth and proliferation |
| | IL-10 | T cell | B cells, | Inhibits cytokine production |
| | | | macrophages | and mononuclear cell |
| | | | | function, anti-inflammatory |
| | IL-11 | BM stromal | B cells | Differentiation, induces acute |
| | | cells | | phase proteins |
| | IL-12 | T cells | NK cells | Activates NK cells |
| Tumor | TNF-α | Macrophages | Macrophages | Phagocyte cell activation, |
| necrosis | | | | endotoxic shock |
| factors | | Monocytes | Tumor cells | Tumor cytotoxicity, cachexia |
| | TNF-β | T-cells | Phagocytes, | Chemotactic, phagocytosis, |
| | | | tumor cells | oncostatic, induces other |
| | | | | cytokines |
| Interferons | IFN-α | Leukocytes | Various | Anti-viral |
| | IFN-β | Fibroblasts | Various | Anti-viral, anti-proliferative |
| | IFN-γ | T-cells | Various | Anti-viral, macrophage |
| | | | | activation, increases |
| | | | | neutrophil and monocyte |
| | | | | function, MHC-I and -II |
| | | | | expression on cells |
| Colony | G-CSF | Fibroblasts, | Stem cells in | Granulocyte production |
| stimulating | | endothelium | BM | |
| factors | GM-CSF | T cells, | Stem cells | Granulocyte, monocyte, |
| | | macrophages, | | eosinophil production |
| | | fibroblasts | | |
| | M-CSF | Fibroblast, | Stem cells | Monocyte production and |
| | | endothelium | | activation |
| | Erythropoietin | Endothelium | Stem cells | Red blood cell production |
| Others | TGF-β | T cells and B | Activated T | Inhibit T and B cell |
| | | cells | | proliferation, inhibit |
| | | | and B cells | hematopoiesis, promote |
| | | | | wound healing |

Abbreviations: BM, bone marrow; DCs dendritic cells; G-CSF, granulocyte-colony stimulating factors; M-CSF, macrophage Colony stimulating factor; Th, T helper cells.

TABLE 4
Functional Classes of Cytokines

| Functional Class | Primary Property | Other Effects | Examples |
|---|---|---|---|
| lymphocyte growth | clonal expansion | Th1/Th2/Th17 | IL-2, IL-4, IL-7, IL-17, IL-15 |
| factors | | polarization | |
| Th1 cytokines | ⬆ Th1 response | clonal expansion of | IFNγ, IL-2, IL-12, IL-18 |
| | | CTL^{a} | |
| Th2 cytokines | ⬆ Th2 responses | ⬆ antibody | IL-4, IL-5, IL-18, IL-25, IL-33 |
| | | production | |
| Th17 cytokines | ⬆ Th17 responses, | autoimmune | IL-17, IL-23, IFNγ |
| | IFNγ | responses | |
| pro-inflammatory | ⬆ inflammatory | ⬆ innate immune | IL-1α, IL-β, TNFα, IL-12, |
| cytokines | mediators | responses | IL-18, IL-23 |
| | | | MIF, IL-32, IL-33, CD40L |
| anti-inflammatory | ↓ inflammatory | ↓ cytokine-mediated | IL-10, IL-13, TGFβ, IL-22, |
| cytokines | genes | lethality | IL-1Ra, IFNα/β |
| adipokines | pro-inflammatory | ↓autoimmune | IL-1α, TNFα, IL-6, leptin, |
| | | disease pro- | adiponectin, resistin |
| | | atherogenic | |
| gp130 signaling | growth factors | B-cell activation, | IL-6, CNTF, IL-11, LIF, CT-1 |
| cytokines | | acute phase | |
| nerve growth factors | ⬆ nerve/Schwann | B-cell activation | BNDF, NGF |
| | cells | | |
| osteoclast activating | bone resorption | immune stimulation | RANK L |
| cytokines | | | |
| colony stimulating | hematopoiesis | pro and anti- | IL-3, IL-7, G-CSF, GM-CSF, |
| factors | | inflammatory | M-CSF |
| angiogenic cytokines | neovascularization | pro-metastatic | VEGF, IL-1, IL-6, IL-8 |
| mesenchymal growth | fibrosis | pro-metastatic | FGF, HGF, TGFβ, BMP |
| factors | | | |
| type II interferon | macrophage | increase class II | IFNγ |
| | activation | MHC | |
| type I interferons | anti-viral; ⬆class I | anti-inflammatory, | IFNα, IFNβ |
| | MHC | anti-angiogenic | |
| chemokines^{b} others | ⬆ cellular | ⬆ cell activation | IL-8, MCP-1, MIP-1α, |
| | emigration | | |

^{α}does not include soluble cytokine receptors such as sTNFRp55, sTNFRp75, sIL-1R type II, IL-18 binding protein, osteoprotegerin
^{b}the chemokine family includes CC and CXC chemokines with over xx members
^{a}CTL, cytotoxic T-cell; BMP, bone morphogenic protein;

Cytokine expression is regulated at the transcription level, translation level, and protein synthesis. The expression of cytokines also seems to be regulated differentially, depending on cell type and developmental age. Secretion or release from the producer cells is a regulated process. Once released, their behavior in the circulation may be regulated by soluble receptors and specific or unspecific binding proteins. Regulation also is at work at the receptor level on target cells and at the level of signaling pathways governing alterations in the behavior of responder cells.

Numerous cytokines are pleiotropic effectors showing multiple biological activities. Multiple cytokines have overlapping activities therefore a single cell frequently interacts with multiple cytokines with seemingly identical responses (cross-talk).

Cytokines show stimulating or inhibitory activities and synergize or antagonize the actions of other factors. The type, the duration, and also the extent of cellular activities induced by a particular cytokine can be influenced considerably by the micro-environment of a cell, depending, for example, on the growth state of the cells (sparse or confluent), the type of neighboring cells, cytokine concentrations, the combination of other cytokines present at the same time, and even on the temporal sequence of several cytokines acting on the same cell. Under such circumstances combinatorial effects thus allow a single cytokine to transmit diverse signals to different subsets of cells.

Although some cytokines are known to share at least some biological effects, the observations that single cells usually show different patterns of gene expression in response to different cytokines can be taken as evidence for the existence of cytokine receptor-specific signal transduction pathways. Shared and different transcriptional activators that transduce a signal from a cytokine receptor to a transcription regulatory element of DNA are involved in these processes such as STAT proteins.

Basic FGF (bFGF) is a strong mitogen for fibroblasts at low concentrations and a chemoattractant at high concentrations. bFGF has been shown also to be a biphasic regulator of human hepatoblastoma-derived HepG2 cells, depending upon the concentration. IFN-γ can stimulate the proliferation of B-cells prestimulated with Anti-IgM, and inhibits the activities of the same cells induced by IL-4. On the other hand, IL-4 activates B-cells and promotes their proliferation while inhibiting the effects induced by IL-2 in the same cells. The activity of at least two cytokines such as IL-1α and IL-1β is regulated by an endogenous receptor antagonist, the IL-1 receptor antagonist. Cytokines, such as TNFα, IFN-γ, IL-2 and IL-4, are inhibited by soluble receptors. Cytokines including IL-10 and TGF-β, inhibit other cytokines.

Thorough examination of the physiological effects of the expression of cytokines in complex organisms has shown that these mediators are involved in all systemic reactions of an organism, including the important processes as regulation of immune responses, for example: BCDF (B-cell growth and differentiation factors), BCGF (B-cell growth factors) TRF (T-cell replacing factors), isotype switching, inflammatory processes, hematopoiesis, and wound healing.

Cytokines play a key role in neuroimmunological, neuroendocrinological, and neuroregulatory processes. Cytokines also regulate cell cycle, differentiation, migration, cell survival and cell death, and cell transformation. Viral infectious agents exploit the cytokine repertoire of organisms to evade immune responses of the host. Virus-encoded factors affect the activities of cytokines in at least four different ways: by inhibiting the synthesis and release of cytokines from infected cells; by interfering with the interaction between cytokines and their receptors; by inhibiting signal transmission pathways of cytokines; and by synthesizing virus-encoded cytokines that antagonize the effects of host cytokines mediating antiviral processes. Bacteria and micro-organisms also appear to produce substances with activities resembling those of cytokines and which they utilize to subvert host responses.

Specific activities of cytokines have been the basis for current concepts of therapeutical intervention, in particular of the treatment of hematopoietic malfunctions and tumor therapy. Applications involve the support of chemo- and radiotherapy, bone marrow transplantation, and general immunostimulation (Justiz Vaillant A A, Qurie A. Interleukin. In: StatPearls. Treasure Island (FL): StatPearls Publishing; 2020 January-. Available from: ncbi.nlm.nih.gov/books/NBK499840/).

Interleukin-1 (IL-1)

Macrophages, large granular lymphocytes, B cells, endothelium, fibroblasts, and astrocytes secrete IL-1. T cells, B cells, macrophages, endothelium and tissue cells are the principal targets. IL-1 causes lymphocyte activation, macrophage stimulation, increased leukocyte/endothelial adhesion, fever due to hypothalamus stimulation, and release of acute phase proteins by the liver. It may also cause apoptosis in many cell types and cachexia.

Interleukin-2 (IL-2)

T cells produce IL-2. The principal targets are T cells. Its primary effects are T-cell proliferation and differentiation, increased cytokine synthesis, potentiating Fas-mediated apoptosis, and promoting regulatory T cell development. It causes proliferation and activation of NK cells and B-cell proliferation and antibody synthesis. Also, it stimulates the activation of cytotoxic lymphocytes and macrophages.

Interleukin-3 (IL-3)

T cells and stem cells make IL-3. It functions as a multilineage colony-stimulating factor.

Interleukin-4 (IL-4)

CD4+ T cells (Th2) synthesize IL-4, and it acts on both B and T cells. It is a B-cell growth factor and causes IgE and IgG1 isotype selection. It causes Th2 differentiation and proliferation, and it inhibits IFN gamma-mediated activation on macrophages. It promotes mast cell proliferation in vivo.

Interleukin-5 (IL-5)

CD4+ T cells (Th2) produce IL-5, and its principal targets are B cells. It causes B-cell growth factor and differentiation and IgA selection. Besides, causes eosinophil activation and increased production of these innate immune cells.

Interleukin-6 (IL-6)

T and B lymphocytes, fibroblasts and macrophages make IL-6. B lymphocytes and hepatocytes are its principal targets. IL-6 primary effects include B-cell differentiation and stimulation of acute phase proteins.

Interleukin-7 (IL-7)

Bone marrow stromal cells produce IL-7 that acts on pre-B cells and T cells. It causes B-cell and T-cell proliferation.

Interleukin-8 (IL-8)

Monocytes and fibroblasts make IL-8. Its principal targets are neutrophils, basophils, mast cells, macrophages, and keratinocytes. It causes neutrophil chemotaxis, angiogenesis, superoxide release, and granule release.

Interleukin-9 (IL-9)

Th9, Th2, Th17, mast cells, NKT cells, and regulatory T cells produce this cytokine. It enhances T-cell survival, mast cell activation and synergy with erythropoietin.

Interleukin-10 (IL-10)

Th2 cells produce IL-10. Its principal targets are Th1 cells. It causes inhibition of IL-2 and interferon gamma. It decreases the antigen presentation, and MHC class II expression of dendritic cells, co-stimulatory molecules on macrophages and it also downregulates pathogenic Th17 cell responses. It inhibits IL-12 production by macrophages.

Interleukin-11 (IL-11)

Bone marrow stromal cells and fibroblasts produce IL-11. The IL-11 principal targets are hemopoietic progenitors and osteoclasts. The IL-11 primary effects include osteoclast formation, colony stimulating factor, raised platelet count in vivo, and inhibition of pro-inflammatory cytokine production.

Interleukin-12 (IL-12)

Monocytes produce IL-12. Its principal targets are T cells. It causes induction of Th1 cells. Besides, it is a potent inducer of interferon gamma production by T lymphocytes and NK cells.

Interleukin-13 (IL-13)

CD4+ T cells (Th2), NKT cells and mast cells synthesize IL-13. It acts on monocytes, fibroblasts, epithelial cells and B cells. The IL-13 significant effects are B-cell growth and differentiation, stimulates isotype switching to IgE. It causes increased mucus production by epithelial cells, increased collagen synthesis by fibroblasts and inhibits pro-inflammatory cytokine production. Also, IL-13 works together with IL-4 in producing biologic effects associated with allergic inflammation and in defense against parasites.

Interleukin-14 (IL-14)

T cells produce IL-14, and its principal effects are stimulation of activated B cell proliferation and inhibition of immunoglobulin secretion.

Interleukin-15 (IL-15)

Monocytes, epithelium, and muscles make IL-15. It acts on T cells and activated B cells. It causes the proliferation of both B and T cells. It causes NK cell memory and CD8+ T cell proliferation.

Interleukin-16 (IL-16)

Eosinophils and CD8+ T cells synthesize IL-16. Its principal target is CD4+ T cells. It causes CD4+ T cell chemoattraction.

Interleukin-17 (IL-17)

This cytokine is produced by Th-17. It acts on epithelial and endothelial cells. IL-17 main effects are the release of IL-6 and other pro-inflammatory cytokines. It enhances the activities of antigen-presenting cells. It stimulates chemokine synthesis by endothelial cells.

Interleukin-18 (IL-18)

Macrophages mostly make IL-18, which can be produced by hepatocytes and keratinocytes. Its principal target is a co-factor in Th1 cell induction. It causes interferon gamma production and enhances NK cell activity.

Interleukin-19 (IL-19)

Th2 lymphocytes synthesize IL-19 and acts on resident vascular cells in addition to immune cells. It is an anti-inflammatory molecule. It promotes immune responses mediated by regulatory lymphocytes and has substantial activity on microvascular.

Interleukin-20 (IL-20)

Immune cells and activated epithelial cells secrete IL-20. It acts on epithelial cells. It plays a vital role in the cellular communication between epithelial cells and the immune system under inflammatory conditions.

Interleukin-21 (IL-21)

NK cells and CD4+ T lymphocytes make IL-21. It acts on various immune cells of innate and the adaptive immune systems. IL-21 promotes B and T lymphocyte proliferation and differentiation. It enhances NK cell activity.

Interleukin-22 (IL-22)

Different cells in both innate and acquired immunities produce IL-22, but the primary sources are T cells. Th22 cell is a new line of CD4+ T cells, which differentiated from naive T cells in the presence of various pro-inflammatory cytokines including IL-6. IL-22 inhibits IL-4 production. It also has essential functions in mucosal surface protection and tissue repair.

Interleukin-23 (IL-23)

Macrophages and dendritic cells mainly synthesize IL-23. It acts on T cells causing maintenance of IL-17 producing T cells.

Interleukin-24 (IL-24)

Monocytes, T and B cells mostly make IL-24. It causes cancer-specific cell death, causes wound healing and protects against bacterial infections and cardiovascular diseases.

Interleukin-25 (IL-25)

Dendritic cells produced predominantly IL-25. It acts on various types of cells, including Th2 cells. It stimulates the synthesis of Th2 cytokine profile including IL-4 and IL-13.

Interleukin-26 (IL-26)

It is strongly associated inflammatory activity with IL-26. Th17 cells produce this interleukin. It acts on epithelial cells and intestinal epithelial cells. It induces IL-10 expression, stimulates the production of IL-1-beta, IL-6, and IL-8 and causes Th17 cell generation.

Interleukin-27 (IL-27)

T cells make IL-27 that activates STAT-1 and STAT-3, which regulates immune responses. IL-27 stimulates IL-10 production. It is a pro-inflammatory molecule and upregulates type-2 interferon synthesis by natural killer cells.

Interleukin-28 (IL-28)

Regulatory T-cells synthesize IL-28, which acts on keratinocytes and melanocytes. It stimulates cell presentation of viral antigens to CD8+T lymphocytes. IL-28 also upregulates TLR-2 and TLR-3 expression. IL-28 enhances the keratinocyte capacity to recognize pathogens in the healthy skin.

Interleukin-29 (IL-29)

IL-29 is a type-3 interferon and produced by virus-infected cells, dendritic cells, and regulatory T-cells. It upregulates viral protective responses. Virus-infected cells may regulate IL-29 genome.

Interleukin-30 (IL-30)

Monocytes mainly produce IL-30 in response to TLR agonists including bacterial LPS. It acts on monocytes, macrophages, dendritic cells, T and B lymphocytes, natural killer cells, mast cells, and endothelial cells.

Interleukin-31 (IL-31)

IL-31 is produced mainly by Th2 cells and dendritic cells. It is a proinflammatory cytokine and a chemotactic factor that direct polymorphonuclear cells, monocytes, and T cells to inflammatory lesions. IL-31 induces chemokines production and synthesis of IL-6, IL-16, and IL-32.

Interleukin-32 (IL-32)

IL-32 is a pro-inflammatory molecule. Natural killer cells and monocytes mainly produce it. IL-32 induces the synthesis of various cytokines including IL-6, and IL-1beta. It inhibits IL-15 production.

Interleukin-33 (IL-33)

Mast cells and Th2 lymphocytes express IL-33 that acts on various innate and immune cells including dendritic cells and T and B lymphocytes. It mediates Th2 responses and therefore participates in the protection against parasites and type-I hypersensitivity reaction.

Interleukin-34 (IL-34)

Various phagocytes and epithelial cells synthesize Interleukin-34 (IL-34). It enhances IL-6 production and participates in the differentiation and development of antigen-presenting cells including microglia.

Interleukin-35 (IL-35)

Regulatory B cells mainly secrete it. One of the primary functions of this interleukin is its involvement in lymphocyte differentiation. It exhibits an immune-suppressive effect.

Interleukin-36 (IL-36)

Phagocytes mainly make IL-36. It acts on T lymphocytes and NK cells regulating the IFN-γ synthesis. It stimulates the hematopoiesis and expression of both MHC class I and II molecules as well as intracellular adhesion molecules (ICAM)-1.

Interleukin-37 (IL-37)

IL-37 plays an essential role in the regulation of the innate immunity causing immunosuppression. Phagocytes and organs including the uterus, testis, and thymus express it. IL-37 upregulates immune responses and inflammation in autoimmune disorders.

Interleukin-38 (IL-38)

11-38 acts on T cells and inhibits the synthesis of IL-17 and IL-22. The placenta, tonsil's B lymphocytes, spleen, skin, and thymus widely express IL-38.

Interleukin (IL-39)

B lymphocytes mainly produce IL-39. It acts on neutrophils inducing their differentiation or expansion.

Interleukin-40 (IL-40)

IL-40 is produced in the bone marrow, fetal liver, and by activated B cells. IL-40 plays a vital role in the development of humoral immune responses.

Accordingly, in certain embodiments, cytokines include lymphokines, monokines, interferons, interleukins (“ILs”), such as IL-1, IL-1α, IL-2, IL-3, IL-4, IL-5, IL-6, IL-7, IL-8, IL-9, IL-10, IL-11, IL-12, IL-13, IL-15, IL-17A-F, IL-18 to IL-29 (such as IL-23), IL-31 to IL-40, including PROLEUKIN™, rIL-2; a tumor-necrosis factor such as TNF-α or TNF-β, TGF-β1-3; and other polypeptide factors including leukemia inhibitory factor (“LIF”), ciliary neurotrophic factor (“CNTF”), CNTF-like cytokine (“CLC”), cardiotrophin (“CT”), and kit ligand (“KL”). As used herein, the term “chemokine” refers to soluble factors (e.g., cytokines) that have the ability to selectively induce chemotaxis and activation of leukocytes. They also trigger processes of angiogenesis, inflammation, wound healing, and tumorigenesis. Example chemokines include IL-8, a human homolog of murine keratinocyte chemoattractant (KC).

Four distinct CD4 T-cell subsets have been shown to exist, T helper 1 (T_{H}1), T helper 2 (T_{H}2), T helper 17 (T_{H}17), and induced T regular (iTreg) cells. They play a critical role in orchestrating adaptive immune responses to various microorganisms. They can be distinguished by their unique cytokine production profiles and their functions: T_{H}1 cells predominantly produce interferon-γ (IFN-γ) and are important for protective immune responses to intracellular viral and bacterial infection; T_{H}2 cells, by producing interleukin-4 (IL-4), IL-5, IL-9, IL-13, and IL-25, are critical for expelling extracellular parasites such as helminths; T_{H}17 cells are responsible for controlling extracellular bacteria and fungi through their production of IL-17a, IL-17f, and IL-22 inducible T-regulatory (iTreg) cells, together with naturally occurring T-regulatory (nTreg) cells, are important in maintaining immune tolerance, as well as in homeostasis, activation and function. Foxp3(+) T-regulatory cells (Tregs) are primarily generated in the thymus (tTreg), but also may be generated extrathymically at peripheral sites (pTreg), or induced in cell culture (iTreg) in the presence of transforming growth factor β (TGF-β).

Type 1 Cytokines: T helper 1 (T_{H}1) cell promoting factors include IFN-γ, IL-12 (p70), and the activation of the transcription factors STAT1 and STAT4. The expression of the interleukin-12 receptor β2-chain (IL-12Rβ2) is required for T_{H}1 cellular differentiation since it allows for the responsiveness to IL-12 on the T_{H}1 cells. IL-12R activation increases IFN-γ expression through STAT1 signals to induce the T_{H}1 master regulator T-bet. This further increases IFN-γ expression while suppressing IL-4. T_{H}1 cells are the primary source for the inflammatory cytokines IFN-γ, IL-2, and TNFβ (LTα). T_{H}1 cytokines stimulate macrophages, lymphocytes, and polymorphonuclear neutrophils (PMN, PML, or PMNL) in the destruction of bacterial pathogens. These cytokines also help foster the development of cytotoxic lymphocytes (CTL) and natural killer (NK) cells that are responsible for the cell-mediated immune response against viruses and tumor cells. Due to the central role of T_{H}1 cells in immune system, over activation or misdirected activation also makes them key players in T_{H}1-dominant autoimmune diseases such as multiple sclerosis, type-1 diabetes, rheumatoid arthritis, and delayed-type hypersensitivity responses.

Type 2 Cytokines: Type 2 immune responses are defined by the cytokines interleukin-4 (IL-4), IL-5, IL-6, IL-9, IL-10 and IL-13, which can either be host protective or have pathogenic activity. Type 2 immunity promotes antihelminth immunity, suppresses type 1-driven autoimmune disease, neutralizes toxins, maintains metabolic homeostasis, and regulates wound repair and tissue regeneration pathways following infection or injury. Nevertheless, when type 2 responses are dysregulated, they can become important drivers of disease. Type 2 immunity induces a complex inflammatory response characterized by eosinophils, mast cells, basophils, type 2 innate lymphoid cells. IL-4- and/or IL-13-conditioned macrophages and T helper 2 (T_{H}2) cells, which are crucial to the pathogenesis of many allergic and fibrotic disorders. As chronic type 2 immune responses promote disease, the mechanisms that regulate their maintenance are thought to function as crucial disease modifiers.

Fusokines: These are artificial combinations of two different cytokines which are genetically linked using a linker sequence. The first example of a fusokine is pIXY321 or pixykine which is a fusion protein of granulocyte-macrophage colony-stimulating factor (GMCSF) and IL-3 that showed superior hematopoietic and immune effects compared to either cytokine alone. GM-CSF-based fusokines with cytokines of the IL-2 family were explored as well. These cytokines all signal through receptor complexes comprising the γc subunit. Examples of such fusokines with GM-CSF include IL-2, IL-15 and IL-21, also known as GIFT2, -15 and -21. Synergistic effects could be expected both at the signaling level (i.e. synergistic effects within a target cell) and cellular level (i.e. synergistic effects between different target cell types). Another example of a fusokine that combines α-helical cytokines is IL-2/IL-12.

Another class of fusokines combines cytokines from different structural families. Examples include the fusion of IL-18 (a member of the IL-1 cytokine family) and IL-2 and the fusion between IL-18 and EGF (epidermal growth factor). Since overexpression of the EGFR is often observed on certain tumor cell types, the latter fusokine offers the possibility to target the IL-18 activity to EGFR⁺ tumor cells. Fusions between α-helical bundle cytokines and chemokines were also explored in greater detail. Chemokines often act using concentration gradients to steer migration of immune cells to sites of infection and inflammation. Many chemokine receptors display a restricted expression pattern allowing targeting to selected (immune) cells. Moreover, signaling via the serpentine, G-protein coupled chemokine receptors is fundamentally different from pathways activated by the α-helical bundle cytokine receptor complexes and synergetic positive and negative cross-talk mechanisms could be expected. Of note, designed N-terminally truncated versions of chemokines can retain their receptor binding properties but display antagonistic behavior. An example is a fusokine between GM-CSF and a N-terminally truncated CCL2 lacking the first 5 N-terminal amino-acids, also known as GMME1. Other examples include fusions between a wild-type cytokine and a mutant cytokine with strongly reduced affinity for its cognate receptor complex

XCL1/IFNα2-Mutant: XCL1 is a 93 amino acids chemokine secreted by CD8⁺ T cells, T_{H}1 cell-polarized CD4⁺ T cells and NK cells. It interacts with XCR1, a chemokine receptor exclusively expressed by dendritic cells. In mice, XCR1 is expressed in the large majority of splenic CD11c⁺ CD8α⁺ dendritic cells whereas only a very minor subset of CD8α⁻ dendritic cells expresses this receptor. XCR1 is a conserved selective marker of mammalian cells (including human cells) homologous to mouse CD8α⁺ dendritic cells.

The human IFNα2-Q124R mutant has a high affinity for the murine IFNAR1 chain and a low affinity for the murine IFNAR2 chain.

CCL20/IL-1β: The CC chemokine CCL20, also known as liver and activation-regulated chemokine (LARC), macrophage inflammatory protein-3α (MIP-3α) or Exodus-1 is a 96 AA protein that is predominantly expressed in liver and lymphoid tissue. Upon secretion, CCL20 exerts its activity by binding to the CC chemokine receptor 6 (CCR6), which belongs to the G-protein coupled receptor (GPCR) 1 family. CCR6 expression is reported on different leukocyte subsets but is best documented for the T_{H}17 cell population. Normal T_{H}17 function is indispensable for protective immunity against a range of pathogens, including Mycobacterium tuberculosis, Klebsiella pneumoniae and Bordetella pertussis.

Potentiating effects of IL-1β on the expansion and differentiation of different T cell subsets, in particular T_{H}17 cells have been firmly established. Among T cell subsets, T_{H}17 cells express the highest levels of the IL-1R and IL-1 plays an important role in T_{H}17 priming. Controlled agonistic IL-1 activity could therefore have applications in different physiological/pathological processes, where immunostimulatory effects would be desirable. One of the main concerns regarding the use of IL-1 in immunostimulatory therapies is however its severe toxicity when administered systemically. Thus, when IL-1 action could be confined to a selected cellular population, the toxicity issue might be resolved, which opens up therapeutic perspectives, e.g. for the use as a T-cell adjuvant to enhance the response to weak vaccines. To specifically target IL-1 mutants to the T_{H}17 cell population, IL-1 variants are used that consist of mutant IL-1 fused to a CCL20 targeting moiety.

TNFα/Leptin Mutant: TNFα is a cytokine with a wide range of biological activities including cytotoxicity, regulation of immune cells and mediation of inflammatory responses. It is a self-assembling, non-covalently bound, homotrimeric type II transmembrane protein of 233 amino acids. TNFα is active as a membrane-bound as well as a soluble protein, released from the cell membrane after proteolytic cleavage of the 76 aminoterminal amino acids (presequence) by TNFα converting enzyme (TACE, also called ADAM17). It signals through 2 distinct receptors, TNF-R1 (p55) and TNF-R2 (p75), both transmembrane glycoproteins with a cystein-rich motif in the ligand-binding extracellular domain. Despite the extracellular homology, they have distinct intracellular domains and therefore signal different TNF activities.

Leptin is a 16 kDa adipocytic cytokine involved in a multitude of biological processes, including immunity, reproduction, linear growth, glucose homeostasis, bone metabolism and fat oxidation, but is best known for its dramatic effect as a satiety signal. Because of its effect on immune cells, leptin is also implicated in several auto-immune diseases. Selective targeting of leptin activity may be beneficial for both metabolic and immune- or inflammation-related disorders.

Modified Cytokines: These cytokines comprise a modified amino acid sequence of the native amino acid sequence of the cytokine. Modifications include mutations, insertions, deletions, modified amino acids, linkers, fusions with other cytokines, antibody binding domains and the like. In certain embodiments the cytokines are modified by chemical groups, for example pegylated interferons. Examples of pegylated interferons include: peginterferon alfa-2b (Peglntron, Sylatron), peginterferon alfa-2a (Pegasys ProClick) and peginterferon beta-1a (Plegridy).

### Cells and Cell Lines

Any type of biological sample can be cultured or preserved in the media embodied herein. The invention encompasses tissues, ex vivo engineered tissues or cells, modified cells, cell lines, pluripotent, multipotent or unipotent cells, purified cell preparations transfected or transformed cells, cell lines, stem cells and purified cell preparations. In some embodiments, the cells, cells lines, stem cells and purified cells preparations of the invention are of mammalian origins, including but not limited to human, primates, rodent, dog, cat, horse, cow, or sheep. In preferred embodiments, they originate from a human. Examples of cells include immune cells, CAR-T cells, stem cells, induced pluripotent stem cells (iPSC), cell lines, germ cells, embryonic stem cells and derivations thereof, adult stem cells and derivations thereof, progenitor cells and derivations thereof, cells derived from mesoderm, endoderm or ectoderm, and a cell of mesoderm, endoderm or ectoderm lineage such as an adipose-derived stem cell, mesenchymal stem cell, hematopoietic stem cell, skin derived precursor cell, hair follicle cell, fibroblast, keratinocyte, epidermal cell, endothelial cell, epithelial cell, granulosa epithelial cell, melanocyte, adipocyte, chondrocyte, hepatocyte, lymphocyte (B and T lymphocyte), granulocyte, macrophage, monocyte, mononuclear cell, pancreatic islet cell, sertoli cell, neuron, glial cell, cardiac muscle cell, and other muscle cell. germ cells, embryonic stem cells and derivations thereof, adult stem cells and derivations thereof, progenitor cells and derivations thereof, cells derived from mesoderm, endoderm or ectoderm, and a cell of mesoderm, endoderm or ectoderm lineage such as an adipose-derived stem cell (ADSC), mesenchymal stem cell, hematopoietic stem cell (CD34⁺ cell), skin derived precursor cell, hair follicle cell, fibroblast, keratinocyte, epidermal cell, endothelial cell, epithelial cell, granulosa epithelial cell, melanocyte, adipocyte, chondrocyte, hepatocyte, lymphocyte (B and T lymphocyte), granulocyte, macrophage, monocyte, mononuclear cell, pancreatic islet cell, sertoli cell, neuron, glial cell, cardiac muscle cell, and other muscle cell.

Examples of immune cells generally includes white blood cells (leukocytes) which are derived from hematopoietic stem cells (HSC) produced in the bone marrow lymphocytes (T cells, B cells, natural killer (NK) cells) and myeloid-derived cells (neutrophil, eosinophil, basophil, monocyte, macrophage, dendritic cells). Among the sub-types and subpopulations of T cells and/or of CD4⁺ and/or of CD8⁺ T cells are naive T (T_{N}) cells, effector T cells (T_{EFF}), memory T cells and sub-types thereof, such as stem cell memory T (T_{SCM}) cells, central memory T (T_{CM}) cells, effector memory T (T_{EM}) cells, or terminally differentiated effector memory T (T_{EMRA}) cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as T_{H}1 cells, T_{H}2 cells, T_{H}3 cells, T_{H}17 cells, T_{H}9 cells, T_{H}22 cells, follicular helper T cells, alpha/beta T cells (αβ T), and gamma/delta T (γδ T) cells.

The cell culture media, preservative or cryopreservative media as described herein and may be used for culturing and/or growing and/or increasing the protein yield of cells such as a vertebrate cell, an arthropod cell, an annelid cell, a molluscs cell, a sponge cell, a jellyfish cell, an insect cell, an avian cell, a mammalian cell, a fish cell, a prokaryotic cell and the like. In some examples, the cell culture media, preservative or cryopreservative media may be used for culturing and/or growing and/or increasing the protein yield of eukaryotic cells or tissues including animal cells, human cells, insect cells, plant cells, avian cells, fish cells, mammalian cells and the like.

In some examples, the mammalian cell may include, but is not limited to, a human cell, a murine cell, a rat cell, a hamster cell, a rabbit cell, a dog cell, a monkey cell, a hybridoma cell, a CHO cell, a CHO-Kl cell, a CHO-DG44 cell, a CHO-S cell, a CHO-DXB11 cell, a CHO-GS cell, a SH87 cell, a BHK cell, a COS cell, a VERO cell, a HeLa cell, a 293 cell, a PER-C6 cell, a K562 cell, a MOLT-4 cell, an M1 cell, an NS-1 cell, a COS-7 cell, an MDBK cell, an MDCK cell, an MRC-5 cell, a WI-38 cell, a WEHI cell, an SP2/0 cell, a CAP cell, a AGE1.HN cell, or a derivative thereof.

### Kits

In certain embodiments, the cell culture media, preservative or cryopreservative media as described herein may be provided as a kit. Thus, in one aspect, there is provided a kit comprising the components of the cell culture media, preservative or cryopreservative media as described herein and the cytokines either as unit doses or premixed.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

What is claimed:

1. A preservation or cryopreservation medium comprising a low dose of one or more: cytokines, hormones, mitogens or combinations thereof.

2. The preservation or cryopreservation medium of claim 1, wherein the medium comprises one or more cytokines, modified cytokines, mutants or derivatives thereof.

3. The preservation or cryopreservation medium of claim 1 or 2, wherein the cytokines comprise one or more type 1 cytokines, type 2 cytokines, type 17 cytokines or combinations thereof.

4. The preservation or cryopreservation medium of claim 3, wherein the type 1 cytokines comprise: interleukin IL-2, IL-12, IL-18, IL-27, interferon-gamma (IFNγ), granulocyte macrophage-colony stimulating factor (GM-CSF), granulocyte colony stimulating factor (G-CSF), leukemia inhibiting factor (LIF), ciliary neurotrophic factor (CNTF), thrombopoietin, tumor necrosis factor alpha (TNFα), tumor necrosis factor β (TNFβ), prolactin or combinations thereof.

5. The preservation or cryopreservation medium of claim 3, wherein the type 2 cytokines comprise: transforming growth factor beta (TGF-β), interleukin IL-4, IL-5, IL-6, IL-9, IL-10, IL-13, IL-25, transforming growth factor beta (TGF-β) or combinations thereof.

6. The preservation or cryopreservation medium of claim 3, wherein the type 17 cytokines comprise: interleukin (IL)-17, IL-21, IL-22 or combinations thereof.

7. The preservation or cryopreservation medium of claim 1, wherein the cytokine is a hematopoietic cytokine comprising: granulocyte macrophage colony stimulating factor (GM-CSF), granulocyte colony stimulating factor (G-CSF), macrophage colony stimulating factor (M-CSF), erythropoietin (EPO), thrombopoietin (TPO), stem cell factor (SCF), stem cell growth factor (SCGF), bone morphogenetic protein (BMP) or combinations thereof.

8. The preservation or cryopreservation medium of claim 1, wherein a low dose comprises from about 0.1 μg to about 10 mg per 100 ml.

9. The preservation or cryopreservation medium of claim 8, wherein a low dose comprises from about 1 μg to about 10 μg per 100 ml.

10. The preservation or cryopreservation medium of claim 8, wherein the preservation or cryopreservation medium comprises an amount of one or more cytokines from about 1×10² International Units/ml to about 1×10⁹ International Units/ml.

11. A cell culture medium comprising a low dose of one or more: cytokines, hormones, mitogens or combinations thereof.

12. The cell culture medium of claim 11, wherein the medium comprises one or more cytokines, modified cytokines, mutants or derivatives thereof.

13. The cell culture medium of claim 11 or 12, wherein the cytokines comprise one or more type 1 cytokines, type 2 cytokines, type 17 cytokines or combinations thereof.

14. The cell culture medium of claim 13, wherein the type 1 cytokines comprise: interleukin IL-2, IL-12, IL-18, IL-27, interferon-gamma (IFNγ), granulocyte macrophage-colony stimulating factor (GM-CSF), granulocyte colony stimulating factor (G-CSF), leukemia inhibiting factor (LIF), ciliary neurotrophic factor (CNTF), thrombopoietin, tumor necrosis factor alpha (TNFα), tumor necrosis factor 13 (TNFβ), prolactin or combinations thereof.

15. The cell culture medium of claim 13, wherein the type 2 cytokines comprise: transforming growth factor beta (TGF-β), interleukin IL-4, IL-5, IL-6, IL-9, IL-10, IL-13, IL-25, transforming growth factor beta (TGF-β) or combinations thereof.

16. The cell culture medium of claim 13, wherein the type 17 cytokines comprise: interleukin (IL)-17, IL-21, IL-22 or combinations thereof.

17. The cell culture medium of claim 11, wherein the cytokine is a hematopoietic cytokine comprising: granulocyte macrophage colony stimulating factor (GM-CSF), granulocyte colony stimulating factor (G-CSF), macrophage colony stimulating factor (M-CSF), erythropoietin (EPO), thrombopoietin (TPO), stem cell factor (SCF), stem cell growth factor (SCGF), bone morphogenetic protein (BMP) or combinations thereof.

18. The cell culture medium of claim 11, wherein a low dose comprises from about 0.1 ng to about 10 mg per 100 ml.

19. The cell culture medium of claim 11, wherein a low dose comprises from about 1 μg to about 10 μg per 100 ml.

20. The cell culture medium of claim 11, wherein the medium comprises an amount of one or more cytokines from about 1×10² International Units/ml to about 1×10⁹ International Units/ml.

21. A preservation or cryopreservation medium comprising a low dose of one or more: cytokines, hormones, mitogens or combinations thereof.

22. The preservation or cryopreservation medium of claim 21, wherein the one or more cytokines comprise: interleukins, chemokines, growth factors, interferons or combinations thereof.

23. The preservation or cryopreservation medium of claim 22, wherein the one or more interleukins comprise: interleukins 1-40 (IL-1 to IL-40) or combinations thereof.

24. The preservation or cryopreservation medium of claim 21, wherein the medium comprises an amount of one or more cytokines from about 1×10² International Units/ml to about 1×10⁹ International Units/ml.
